(19) United States
(12) Patent Application Publication
(10) Pub. No.: US 2022/0090218 A1
(43) Pub. Date: Mar. 24, 2022

(54) COMPOSITIONS AND METHODS FOR DETECTING HUMAN PAPILLOMAVIRUS

(71) Applicant: Hangzhou New Horizon Health Technology Co. Ltd., Binjiang District, Hangzhou, Zhejiang (CN)

(72) Inventors: Yang WU, Beijing (CN); Gang LIU, Beijing (CN); Ning LU, Hangzhou, Zhejiang (CN); Yiyou Chen, Hangzhou, Zhejiang (CN)

(21) Appl. No.: 17/420,442

(22) PCT Filed: Jan. 3, 2020
(86) PCT No.: PCT/CN2020/070287

§ 371 Date: Jul. 2, 2021

(30) Foreign Application Priority Data
Jan. 3, 2019 (CN) PCT/CN2019/070277

(51) Int. Cl. C12Q 1/70

(52) U.S. Cl. CPC C12Q 1/708; C12Q 2600/16; C12Q 2600/158

(57) ABSTRACT

The present disclosure relates to compositions and methods for detecting and/or identifying the genotype of a human papillomavirus (HPV) in a biological sample obtained from a subject in need thereof. Particularly, the present disclosure provides primers, probes and kits for simultaneously detecting multiple HPV genotypes in a single-tube PCR reaction.

### CROSS-REFERENCE

This application claims the benefit of the PCT Application No. PCT/CN2019/070277, filed Jan. 3, 2019, which application is incorporated herein by reference in its entirety.

### FIELD OF THE INVENTION

The present disclosure relates to compositions and methods for detecting and/or genotyping human papillomavirus (HPV).

### BACKGROUND OF THE INVENTION

Human papillomavirus (HPV) is a virus of the papillomavirus genera of the papovavirus family. It has host specificity and tissue specificity and normally infects human skin and mucosal cells. It is a common pathogen that is mainly transmitted through sexual activity.

The HPV genome is a double-stranded DNA that forms a closed loop. The genome can be divided into three regions, namely the early region (E region), the late region (L region), and the non-coding region (NCR). E region can be further divided into seven open reading frames (E1 to E7), which mainly encode proteins involved in viral replication, transcription, regulation and cell transformation. L region can be divided into L1 and L2, which encode the major capsid protein and the minor capsid protein, respectively. In total more than 200 subtypes of HPV have been discovered. These subtypes are classified into high-risk types and low-risk types based on their virulence to genital tract-related tumors and carcinogenic risk. Common low-risk subtypes such as HPV6, HPV11, HPV42, HPV43, and HPV44, etc. often cause benign lesions such as external genital warts, while high-risk subtypes HPV16, HPV18, HPV31, HPV33, HPV35, HPV39, HPV45, HPV51, HPV52, HPV56, HPV58, HPV59, and HPV68, etc. can cause cancers such as cervical cancer and cervical intraepithelial neoplasia. In addition, HPV26, HPV53, and HPV66 are suspected high-risk subtypes. Studies have shown that HPV is detectable in 99% of patients with cervical cancer. In 1995, the International Agency for Research on Cancer (IARC) Symposium concluded that HPV infection is the leading cause of cervical cancer. Certain subtypes of HPV can also cause anal cancer, oropharyngeal cancer, vulvar and vaginal cancer, and penile cancer. Most (70%-90%) HPV infections are asymptomatic and are automatically cleared by the immune system within 1-2 years. Therefore, regular HPV testing can effectively prevent the occurrence of related cancers.

HPV detection technology includes the traditional cytology methods, the widely used HPV DNA detection methods and the latest HPV mRNA detection methods. The traditional cytology detection methods, such as Pap smear and liquid-based cytology, have low sensitivity and poor specificity. Although HPV mRNA detection methods have better specificity for detection of cervical lesions after CIN2 (cervical intraepithelial neoplasia stage 2), the methods use mRNA as a detection target, which has higher requirements for clinical sample collection and preservation, and nucleic acid extraction.

Currently, the most widely used HPV DNA detection technology can be divided into the following categories: (1) after PCR amplification using MY9/11, PGMY09/11, GP5+/6+ or other universal primers, hybridization of a specific probe is used for detection, such as PCR-reverse dot blotting, and Gene chip method, etc.; (2) signal amplification method, such as HC2 HPV DNA test (Digene) and Cervista™ HPV HR (Hologic), etc., (3) real-time fluorescent quantitative PCR, such as Cobas® 4800 HPV detection method (Roche).

The method based on hybridization detection after PCR amplification using universal primer PCR has the disadvantages of cumbersome operation, frequent cross-contamination and inconsistent amplification efficiency of different HPV subtypes. The HC2 and Cervista™ techniques based on signal amplification have the disadvantages of high limit of detection (LOD) which leads to low sensitivity.

The Cobas® 4800 HPV test, which is based on a fluorescence real-time PCR, was approved by the FDA for screening for cervical cancer in 2014, but the method requires a dedicated instrument and is costly. In addition, Bernal et al. (Journal of Clinical Virology 61:548-552 (2014)) reported that the when urine samples and cervical samples derived from the same patient population were analyzed by the Cobas® 4800 HPV test, the overall percent agreement between HPV detection in urine and cervical samples was only 88%, indicating that there were significant misdetections when urine samples were used.

In addition, clinical test samples used in the above-mentioned detection methods are normally cervical exfoliated cell samples collected using cervical swabs or sampling brushes. This sampling method is invasive, which may cause pain and discomfort, and not preferred by women in conservative countries/regions due to cultural and/or religion reasons. Particularly, due to traditional moral and ethical restrictions in certain regions, the invasive sampling method used in the above-mentioned detection methods is not acceptable to females, especially unmarried females. This greatly reduces the willingness of females to take the HPV testing, thus limits the HPV test participation rate. Meanwhile, males can also carry HPV, so detection of HPV in male population has a positive effect on preventing the occurrence of cervical cancer in the female population. However, in the current clinical practice, there are few HPV tests for men, and the common sampling method for men (i.e., urethral swabs) is very painful. Thus, there is still a great need for new HPV test methods that are less invasive, easy and cheap to perform, without compromising accuracy, sensitivity, and specificity. The present disclosure provides HPV detection methods that use urine samples for HPV DNA detection, so that the sample collection can be achieved in a non-invasive, painless, quick and convenient way. The present disclosure also provides a full set of compositions and methods for urine DNA extraction and purification, and for the detection of fourteen high-risk HPV subtypes and at least two low-risk HPV subtypes. The invention described herein not only increases the participation rate of female population women in HPV testing, but is also very suitable for HPV testing in the male population. Thus, the invention has great social significance for the prevention of HPV related conditions, such as cervical cancer.

### SUMMARY OF THE INVENTION

The present disclosure provides primers and probes related to HPVs. In some embodiments, these primers and probes can be used for detecting and/or identifying a genotype of a human papillomavirus (HPV). In some embodiments, provided are a combination of primers and probes comprises one or more groups of primer and probe selected from the groups consisting of:

(1) a forward primer comprising the polynucleotide sequence having at least 85%, 90%, 95%, or 100% identity to the sequence of SEQ ID NO: 1, a reverse primer comprising the polynucleotide sequence having at least 85%, 90%, 95%, or 100% identity to the sequence of SEQ ID NO: 2, and a probe comprising the polynucleotide sequence having at least 85%, 90%, 95%, or 100% identity to the sequence of SEQ ID NO: 37;
(2) a forward primer comprising the polynucleotide sequence having at least 85%, 90%, 95%, or 100% identity to the sequence of SEQ ID NO: 3, a reverse primer comprising the polynucleotide sequence having at least 85%, 90%, 95%, or 100% identity to the sequence of SEQ ID NO: 4, and a probe comprising the polynucleotide sequence having at least 85%, 90%, 95%, or 100% identity to the sequence of SEQ ID NO: 38;
(3) a forward primer comprising the polynucleotide sequence having at least 85%, 90%, 95%, or 100% identity to the sequence of SEQ ID NO: 5, a reverse primer comprising the polynucleotide sequence having at least 85%, 90%, 95%, or 100% identity to the sequence of SEQ ID NO: 6, and a probe comprising the polynucleotide sequence having at least 85%, 90%, 95%, or 100% identity to the sequence of SEQ ID NO: 39;
(4) a forward primer comprising the polynucleotide sequence having at least 85%, 90%, 95%, or 100% identity to the sequence of SEQ ID NO: 7, a reverse primer comprising the polynucleotide sequence having at least 85%, 90%, 95%, or 100% identity to the sequence of SEQ ID NO: 8, and a probe comprising the polynucleotide sequence having at least 85%, 90%, 95%, or 100% identity to the sequence of SEQ ID NO: 39;
(5) a forward primer comprising the polynucleotide sequence having at least 85%, 90%, 95%, or 100% identity to the sequence of SEQ ID NO: 9, a reverse primer comprising the polynucleotide sequence having at least 85%, 90%, 95%, or 100% identity to the sequence of SEQ ID NO: 10, and a probe comprising the polynucleotide sequence having at least 85%, 90%, 95%, or 100% identity to the sequence of SEQ ID NO: 39;
(6) a forward primer comprising the polynucleotide sequence having at least 85%, 90%, 95%, or 100% identity to the sequence of SEQ ID NO: 11, a reverse primer comprising the polynucleotide sequence having at least 85%, 90%, 95%, or 100% identity to the sequence of SEQ ID NO: 12, and a probe comprising the polynucleotide sequence having at least 85%, 90%, 95%, or 100% identity to the sequence of SEQ ID NO: 40;
(7) a forward primer comprising the polynucleotide sequence having at least 85%, 90%, 95%, or 100% identity to the sequence of SEQ ID NO: 13, a reverse primer comprising the polynucleotide sequence having at least 85%, 90%, 95%, or 100% identity to the sequence of SEQ ID NO: 14, and a probe comprising the polynucleotide sequence having at least 85%, 90%, 95%, or 100% identity to the sequence of SEQ ID NO: 41;
(8) a forward primer comprising the polynucleotide sequence having at least 85%, 90%, 95%, or 100% identity to the sequence of SEQ ID NO: 15, a reverse primer comprising the polynucleotide sequence having at least 85%, 90%, 95%, or 100% identity to the sequence of SEQ ID NO: 16, and a probe comprising the polynucleotide sequence having at least 85%, 90%, 95%, or 100% identity to the sequence of SEQ ID NO: 42;
(9) a forward primer comprising the polynucleotide sequence having at least 85%, 90%, 95%, or 100% identity to the sequence of SEQ ID NO: 17, a reverse primer comprising the polynucleotide sequence having at least 85%, 90%, 95%, or 100% identity to the sequence of SEQ ID NO: 18, and a probe comprising the polynucleotide sequence having at least 85%, 90%, 95%, or 100% identity to the sequence of SEQ ID NO: 42;
(10) a forward primer comprising the polynucleotide sequence having at least 85%, 90%, 95%, or 100% identity to the sequence of SEQ ID NO: 19, a reverse primer comprising the polynucleotide sequence having at least 85%, 90%, 95%, or 100% identity to the sequence of SEQ ID NO: 20, and a probe comprising the polynucleotide sequence having at least 85%, 90%, 95%, or 100% identity to the sequence of SEQ ID NO: 41;
(11) a forward primer comprising the polynucleotide sequence having at least 85%, 90%, 95%, or 100% identity to the sequence of SEQ ID NO: 21, a reverse primer comprising the polynucleotide sequence having at least 85%, 90%, 95%, or 100% identity to the sequence of SEQ ID NO: 22, and a probe comprising the polynucleotide sequence having at least 85%, 90%, 95%, or 100% identity to the sequence of SEQ ID NO: 39;
(12) a forward primer comprising the polynucleotide sequence having at least 85%, 90%, 95%, or 100% identity to the sequence of SEQ ID NO: 23, a reverse primer comprising the polynucleotide sequence having at least 85%, 90%, 95%, or 100% identity to the sequence of SEQ ID NO: 24, and a probe comprising the polynucleotide sequence having at least 85%, 90%, 95%, or 100% identity to the sequence of SEQ ID NO: 40;
(13) a forward primer comprising the polynucleotide sequence having at least 85%, 90%, 95%, or 100% identity to the sequence of SEQ ID NO: 25, a reverse primer comprising the polynucleotide sequence having at least 85%, 90%, 95%, or 100% identity to the sequence of SEQ ID NO: 26, and a probe comprising the polynucleotide sequence having at least 85%, 90%, 95%, or 100% identity to the sequence of SEQ ID NO: 41;
(14) a forward primer comprising the polynucleotide sequence having at least 85%, 90%, 95%, or 100% identity to the sequence of SEQ ID NO: 27, a reverse primer comprising the polynucleotide sequence having at least 85%, 90%, 95%, or 100% identity to the sequence of SEQ ID NO: 28, and a probe comprising the polynucleotide sequence having at least 85%, 90%, 95%, or 100% identity to the sequence of SEQ ID NO: 40;
(15) a forward primer comprising the polynucleotide sequence having at least 85%, 90%, 95%, or 100% identity to the sequence of SEQ ID NO: 29, a reverse primer comprising the polynucleotide sequence having at least 85%, 90%, 95%, or 100% identity to the sequence of SEQ ID NO: 30, and a probe comprising the polynucleotide sequence having at least 85%, 90%, 95%, or 100% identity to the sequence of SEQ ID NO: 40
(16) a forward primer comprising the polynucleotide sequence having at least 85%, 90%, 95%, or 100% identity to the sequence of SEQ ID NO: 33, a reverse primer comprising the polynucleotide sequence having at least 85%, 90%, 95%, or 100% identity to the sequence of SEQ ID NO: 34, and a probe comprising the polynucleotide sequence having at least 85%, 90%, 95%, or 100% identity to the sequence of SEQ ID NO: 44;
(17) a forward primer comprising the polynucleotide sequence having at least 85%, 90%, 95%, or 100% identity to the sequence of SEQ ID NO: 35, a reverse primer comprising the polynucleotide sequence having at least 85%, 90%, 95%, or 100% identity to the sequence of SEQ ID NO: 36, and a probe comprising the polynucleotide sequence having at least 85%, 90%, 95%, or 100% identity to the sequence of SEQ ID NO: 45, and any combination thereof.

In some embodiments, the combination of primer and probe comprises the primers and probes in (1) and (2); the primers and probes in (3), (4), (5), and (11); the primers and probes in (6), (12), (14), and (15); the primers and probes in (7), (10), and (13); and/or the primers and probes in (8) and (9).

In some embodiments, the combination of primer and probe comprises: at least one group of primers and probes in (1) and (2) and at least one group of primers and probes in (3), (4), (5), and (11).

In some embodiments, the combination of primer and probe comprises: at least one group of primers and probes in (1) and (2) and at least one group of primers and probes in (6), (12), (14), and (15).

In some embodiments, the combination of primer and probe comprises: at least one group of primers and probes in (1) and (2) and at least one group of primers and probes in (7), (10), and (13).

In some embodiments, the combination of primer and probe comprises: at least one group of primers and probes in (1) and (2) and at least one group of primers and probes in (8) and (9).

In some embodiments, the combination of primer and probe comprises: at least one group of primers and probes in (1) and (2), at least one group of primers and probes in (3), (4), (5), and (11), and at least one group of primers and probes in (6), (12), (14), and (15).

In some embodiments, the combination of primer and probe comprises: at least one group of primers and probes in (1) and (2), at least one group of primers and probes in (3), (4), (5), and (11), and at least one group of primers and probes in (7), (10), and (13).

In some embodiments, the combination of primer and probe comprises: at least one group of primers and probes in (1) and (2), at least one group of primers and probes in (3), (4), (5), and (11), and at least one group of primers and probes in (8) and (9).

In some embodiments, the combination of primer and probe comprises: at least one group of primers and probes in (1) and (2), at least one group of primers and probes in (6), (12), (14), and (15), and at least one group of primers and probes in (7), (10), and (13).

In some embodiments, the combination of primer and probe comprises: at least one group of primers and probes in (1) and (2), at least one group of primers and probes in (6), (12), (14), and (15), and at least one group of primers and probes in (8) and (9).

In some embodiments, the combination of primer and probe comprises: at least one group of primers and probes in (1) and (2), at least one group of primers and probes in (7), (10), and (13), and at least one group of primers and probes in (8) and (9).

In some embodiments, the combination of primer and probe comprises: at least one group of primers and probes in (1) and (2), at least one group of primers and probes in (3), (4), (5), and (11), at least one group of primers and probes in (6), (12), (14), and (15), and at least one group of primers and probes in (7), (10), and (13).

In some embodiments, the combination of primer and probe comprises: at least one group of primers and probes in (1) and (2), at least one group of primers and probes in (3), (4), (5), and (11), at least one group of primers and probes in (6), (12), (14), and (15), and at least one group of primers and probes in (8) and (9).

In some embodiments, the combination of primer and probe comprises: at least one group of primers and probes in (1) and (2), at least one group of primers and probes in (3), (4), (5), and (11), and at least one group of primers and probes in (7), (10), and (13), and at least one group of primers and probes in (8) and (9).

In some embodiments, the combination of primer and probe comprises: at least one group of primers and probes in (1) and (2), at least one group of primers and probes in (6), (12), (14), and (15), and at least one group of primers and probes in (7), (10), and (13), and at least one group of primers and probes in (8) and (9).

In some embodiments, the combination of primer and probe described herein further comprises the group of primers and probes in (16) and/or (17).

In some embodiments, the combination of primer and probe consists of the group of primers and probes in (1) to (15), or consists of the group of primers and probes in (1) to (17).

In some embodiments, the combination of primer and probe further comprises a group of primer and probe for a reference control gene. In some embodiments, the reference control gene is β-Actin (ACTB), wherein the primers for the ACTB gene are SEQ ID NOs: 31 and 32, and the probe for the ACTB gene is SEQ ID NO: 43.

In some embodiments, each probe as described herein has a fluorescent dye attached to 5′ end thereof. In some embodiments, the fluorescent dye is selected from the group consisting of FAM (fluorescein), TET, JOE, VIC (2′-chloro-7′phenyl-1,4-dichloro-6-carboxy-fluorescein), HEX (Hexachloro-fluorescein), ROX, TAMRA, Cy3, cy3.5, Cy5, Cy5.5, OregonGreen™, CALRed™, Red640, Texas Red, LighterCycler®Cyan500, LighterCycler®, Red610, a biotin binding material, Alexa 647, Alexa 555, 5-(2-aminoethyl)amino-1-naphthalene sulfonic acid (EDANS), tetramethyl rhodamine (TMR), tetramethyl rhodamine isocyanate (TMRITC), fluorescein isocyanate (FITC), and χ-rhodamine.

In some embodiments, the fluorescent dye on the probes in (3), (4), (5), and/or (11) is the same dye or different dyes having about the same emission wavelength. In some embodiments, the fluorescent dye on the probes in (6), (12), (14), and/or (15) is the same dye, or different dyes having about the same emission wavelength. In some embodiments, the fluorescent dye on the probes in (7), (10), and/or (13) is the same dye, or different dyes having about the same emission wavelength. In some embodiments, the fluorescent dye on the probes in (8) and (9) is the same dye, or different dyes having about the same emission wavelength. In some embodiments, the fluorescent dye on the probes in (3), (4), (5), and/or (11) are different dyes having about the same or different emission wavelengths. In some embodiments, the fluorescent dye on the probes in (6), (12), (14), and/or (15) are different dyes having about the same or different emission wavelengths. In some embodiments, the fluorescent dye on the probes in (7), (10), and/or (13) are different dyes having about the same or different emission wavelengths. In some embodiments, the fluorescent dye on the probes in (8) and (9) are different dyes having about the same or different emission wavelengths.

In some embodiments, each probe has a fluorescent dye attached to 5′ end thereof, and wherein:
(i) the probe in (1) has a first dye;
(ii) the probe in (2) has a second dye;
(iii) the probes in (3), (4), (5), and (11) have a third dye;
(iv) the probes in (6), (12), (14) and (15) have a fourth dye;
(v) the probes in (7), (10), and (13) have a fifth dye; and
(vi) the probes in (8) and (9) have a sixth dye, wherein the dye in (iii) to (vi) are the same dye, but different from the dye in (i) and (ii).

In some embodiments, each probe has a fluorescent dye attached to 5′ end thereof, and wherein:
(i) the probe in (1) has a first dye;
(ii) the probe in (2) has a second dye;
(iii) the probes in (3), (4), (5), and (11) have a third dye;
(iv) the probes in (6), (12), (14) and (15) have a fourth dye;
(v) the probes in (7), (10), and (13) have a fifth dye;
(vi) the probes in (8) and (9) have a sixth dye,
(vii) the probe in (16) has a seventh dye; and
(viii) the probe in (17) has an eighth dye; wherein
the dye in (iii) to (vi) are the same dye, but different from the dye in (i), (ii), (vii) and (viii).

In some embodiments, the combination of primers and probes further comprises a group of primers and probe for a reference control gene, wherein the probe for the reference control gene also has a fluorescent dye attached to 5′ end thereof, and the fluorescent dye for the control gene is different from other dyes in the combination.

In some embodiments, each probe described herein has a fluorescent quencher attached to 3′ end thereof. In some embodiments, the fluorescent quencher is selected from the group consisting of DDQ-I, DDQ-II, Dabcyl, Eclipse, Iowa Black FQ, Iowa Black RQ, BHQ-1, BHQ-2, BHQ-3, QSY-7, QSY-9, and QSY-21.

In some embodiments, the probe in (1) comprises a Cy5 fluorescent dye attached to its 5′ end, and a BHQ-2 fluorescent quencher attached to its 3′ end; the probe in (2) comprises a FAM fluorescent dye attached to its 5′ end, and a BHQ-1 fluorescent quencher attached to its 3′ end; the probes in (3) to (15) comprise a VIC fluorescent dye attached to their 5′ ends, and a MGBNFQ fluorescent quencher attached to their 3′ ends; and the probes in (16) and (17) comprises a FAM fluorescent dye attached to their 5′ ends, and a BHQ-1 fluorescent quencher attached to their 3′ ends.

In some embodiments, the probe in (1) comprises a Cy5 fluorescent dye attached to its 5′ end, and a BHQ-2 fluorescent quencher attached to its 3′ end; the probe in (2) comprises a FAM fluorescent dye attached to its 5′ end, and a BHQ-1 fluorescent quencher attached to its 3′ end; the probes in (3) to (15) comprise a VIC fluorescent dye attached to their 5′ ends, and a MGBNFQ fluorescent quencher attached to their 3′ ends; the probes in (16) and (17) comprises a FAM fluorescent dye attached to their 5′ ends, and a BHQ-1 fluorescent quencher attached to their 3′ ends, and the probe for the reference control gene comprises a ROX fluorescent dye attached to its 5′ end, and a BHQ-2 fluorescent quencher attached to its 3′ end.

The present disclosure also provides compositions comprising the combination of primer and probe as described herein.

The present disclosure also provides DNA chips for detecting and/or genotyping HPVs. In some embodiments, the DNA chips comprise one or more polynucleotide sequence selected from SEQ ID NOs: 1 to 45.

The present disclosure also provides kits for detecting and/or identifying the genotype of a human papillomavirus (HPV) in a biological sample, comprising a combination of primer and probe as described herein, PCR buffer, dNTP, MgCl2, PCR additive, Taq enzyme, and negative control and positive control as quality control.

In some embodiments, kits used to detect and/or identify the human papillomavirus (HPV) genotype in biological samples include: HPV qPCR mixture, Taq enzyme, negative control, and positive control.

In some embodiments, the HPV qPCR mixture comprises a primer and probe combination as described herein, a PCR buffer, dNTP, MgCl₂, PCR additives, and deionized water. In some embodiments, primer and probe combinations include all primer probe combinations in (1)˜(15) and reference control gene primers and probes (SEQ ID NOs: 31, 32, and 43) with primer concentrations of 0.1 μM˜1.2 μM and probe concentrations of 1/5˜1 times the corresponding primer concentrations. In some embodiments, the PCR buffer contains approximately 10-30 mM Tris-HCL buffer and approximately 30˜70 mM KCL, preferably with a Tris-HCL buffer concentration of approximately 20.5 mM and a preferred KCL concentration of approximately 51 mM. In some embodiments, dNTP concentration is 0.15 mM˜0.3 mM, preferably approximately 0.25 mM. In some embodiments, the concentration of MgCl₂ is 1.5 mm˜4 mM, preferably approximately 3.0 mM. In some embodiments, PCR additives contains about 0.1˜1 mg/ml BSA, 0.2%˜2% (V/V) formamide, 0.2 mM˜2 mM spermine, 10 mM˜30 mM tetramethyl ammonium chloride, 0.01 mM˜0.1 mM dithiothreitol (DTT), 0.2%˜2% 2-pyrrolidone, preferred PCR additives contains about 0.64 mg/ml of BSA, about 1% (V/V) formamide, about 1 mM spermine, about 21 mM tetramethyl ammonium chloride, about 0.064 mM DTT, and about 1% (V/V) 2-pyrrolidone.

In some embodiments, the Taq enzyme contains a Platinum™ Taq DNA Polymerase (Invitrogen™, 10966018) with a concentration of 1˜6 U/μl, and preferably a Platinum™ Taq DNA Polymerase (Invitrogen™, 10966018) with a concentration of 4 U/μl.

In some embodiments, the negative control is obtained from urine or its DNA diluted 1-1000 times in adults with high risk HPV DNA negative, preferably by approximately 100 times.

In some embodiments, the positive control is a plasmid containing the high risk HPV L1 gene with a final concentration of 10˜10⁵ copies/μl prepared using the negative control as diluent. The high risk HPV L1 genotype may be one or more of the 14 high risk HPV genotypes described in the invention. Preferably, the positive control contains L1 plasmids of HPV16, HPV18 and HPV45, and their final concentrations are 10³ copies/μl.

The invention also provides a kit for detecting and/or identifying the (HPV) genotype of human papillomavirus in biological samples and capable of guiding and evaluating the efficacy of HPV vaccine, including a combination of primers and probes, PCR buffer, dNTP, MgCl₂, PCR additive, Taq enzyme, and negative control and positive control as quality control.

In some embodiments, the kit used to detect and/or identify human papilloma virus (HPV) genotypes in biological sample for HPV vaccine guidelines and efficacy evaluation includes HPV qPCR mixture I, HPV qPCR mixture II, Taq enzyme, as well as negative control and positive control as quality control.

In some embodiments, HPV qPCR mixture I includes primer and probe combinations mentioned in the present invention, the PCR buffer, dNTP, MgCl₂, PCR additives, and deionized water. In some embodiments, primer and probe combinations include all primer probe combinations in (1), (2), (5), (6), (8), (10), (12), (13), (14), and (15), as well as reference control gene primers and probes (SEQ ID NOs: 31, 32, and 43), with primer concentrations ranging from 0.204 to 1.204 and probe concentrations ranging from 1/5 to 1 times the corresponding primer concentrations. In some embodiments, the PCR buffer contains approximately 10˜30 mM Tris-HCl buffer and approximately 30˜70 mM KCl, preferably with a Tris-HCl buffer concentration of approximately 25.6 mM and a preferred KCl concentration of approximately 64.1 mM. In some embodiments, dNTP concentration is 0.15 mM˜0.3 mM, preferably approximately 0.25 mM. In some embodiments, the concentration of MgCl₂ is 1.5 mm˜4 mM, preferably approximately 3.0 mM. In some embodiments, the PCR additives contain approximately 0.1˜1 mg/ml BSA, 0.2%˜2% (V/V) formamide, 0.2˜2 mM spermidine, 10 mM˜30 mM tetramethylammonium chloride, 0.01 mM˜0.1 mM DTT, and 0.2%˜2% 2-pyrrolidone. Preferably, the PCR additives contains approximately 0.64 mg/ml BSA, approximately 1% (V/V) formamide, approximately 1 mM spermidine, approximately 21 mM tetramethylammonium chloride, approximately 0.064 mM DTT, approximately 1% (V/V) 2-pyrrolidone.

In some embodiments, HPV qPCR mixture II including prime and probe combinations mentioned in the present invention, PCR buffer, dNTP, MgCl₂, PCR additive, and deionized water. In some embodiments, primer and probe combinations including all primer probe combinations in (3), (4), (7), (9), (11), (16), (17), and reference control gene primers and probes (SEQ ID NOs: 31, 32, and 43), with primer concentration of 0.2 μM˜1.2 μM and probe concentrations ranging from 1/5 to 1 times the corresponding primer concentrations. In some embodiments, the PCR buffer contains approximately 10˜30 mM Tris-HCl buffer and approximately 3070 mM KCl, preferably with a Tris-HCl buffer concentration of approximately 25.6 mM, preferably with a KCl concentration of approximately 64.1 mM. In some embodiments, the dNTP concentration is 0.15 mM˜3 mM, preferably with a dNTP concentration of approximately 0.25 mM. In some embodiments, the concentration of MgCl₂ is 1.5 mm˜4 mM, preferably approximately 3.0 mM. In some embodiments, the PCR additive contains approximately 0.1˜1 mg/ml BSA, 0.2%-2% (V/V) formamide, 0.2˜2 mM spermidine, 10 mM˜30 mM tetramethylammonium chloride, 0.01 mM˜0.1 mM DTT, and 0.2%˜2% 2-pyrrolidone. Preferably, PCR additives contain approximately 0.64 mg/ml BSA, approximately 1% (V/V) formamide, approximately 1 mM spermidine, approximately 21 mM tetramethylammonium chloride, approximately 0.064 mM DTT, and approximately 1% (V/V) 2-pyrrolidone.

In some embodiments, the biological sample is collected from a human subject. In some embodiments, the biological sample comprises urine of the human subject.

In some embodiments, the kit further comprises reagents for isolating DNA from the biological sample.

In some embodiments, the reagents for isolating DNA from the biological sample comprises: a lysis solution, magnetic nanoparticles, a protease, a first washing buffer, a second washing buffer, an elution buffer, or any combination thereof.

In some embodiments, the lysis solution comprises guanidinium isothiocyanate, Triton X 100, Tris-HCl, EDTA, and isopropanol. In some embodiments, the guanidinium isothiocyanate has a concentration of about 2 to 6 M. In some embodiments, the Triton X-100 has a concentration of about 1 to 5%. In some embodiments, the Tris-HCl has a concentration of about 20 to 50 mM, wherein the lysis solution has a pH of about 6.5. In some embodiments, the EDTA has a concentration of about 10 to 50 mM. In some embodiments, isopropanol is added after all other components are mixed together. In some embodiments, the isopropanol has a dosage of about 50% to 200% (v/v).

In some embodiments, the lysis solution comprises guanidinium isothiocyanate, Triton X 100, Tris-HCl, EDTA, and isopropanol. In some embodiments, the guanidinium isothiocyanate has a concentration of about 1 to 2 M. In some embodiments, the Triton X-100 has a concentration of about 1 to 2%. In some embodiments, the Tris-HCl has a concentration of about 5 to 10 mM. In some embodiments, the lysis solution has a pH of about 6-7. In some embodiments, the EDTA has a concentration of about 3 to 5 mM. In some embodiments, the isopropanol has a volume of about 50% to 80% (v/v) of the lysis solution.

In some embodiments, the magnetic nanoparticles have an inner core layer and an outer shell layer, and the inner core layer is composed of core-shell type magnetic nanoparticles, wherein the outer shell layer is composed of SiO₂. In some embodiments, the magnetic nanoparticles have a diameter of about 100 to 1000 nm, and a concentration of about 50 mg/ml.

In some embodiments, the first washing buffer comprises guanidinium isothiocyanate, Tris-HCl, NaCl, and ethanol. In some embodiments, the guanidinium isothiocyanate has a concentration of about 50 mM. In some embodiments, the Tris-HCl has a concentration of about 20 to 50 mM. In some embodiments, the first washing buffer has a pH of about 5.0. In some embodiments, the NaCl has a concentration of about 50 to 200 mM. In some embodiments, the ethanol has concentration of about 40% to 60% (v/v).

In some embodiments, the second washing buffer comprises Tris-HCl and ethanol. In some embodiments, the Tri-HCl in the second washing buffer has a concentration of about 10 to 50 mM, and the second washing buffer has a pH of about 6.0. In some embodiments, the ethanol has concentration of about 70% to 80% (v/v).

In some embodiments, the elution buffer is a Tris-EDTA buffer having a pH of about 8.0.

In some embodiments, the protease is protease K. In some embodiments, the protease K has a concentration of about 10 to 20 mg/ml.

The present disclosure further provides methods for detecting and/or identifying the genotype of a human papillomavirus (HPV) in a biological sample obtained from a subject in need thereof. In some embodiments, the methods comprise: (a) obtaining DNA from the biological sample; (b) amplifying the DNA by a fluorescent PCR using a combination of primer and probe as described herein; and (c) determining the presence or absence of DNA of one or more HPV subtype in the biological sample based on the results of the fluorescent PCR.

The present disclosure further provides methods for detecting and/or identifying the genotype of a human papillomavirus (HPV) in a biological sample obtained from a subject in need thereof. In some embodiments, the methods comprise (a) obtaining DNA from the biological sample; (b) amplifying the DNA by a fluorescent PCR using the kit as described herein; and (c) determining the presence or absence of DNA of one or more HPV subtype in the biological sample based on the results of the fluorescent PCR.

The present disclosure further provides methods for detecting and/or identifying the genotype of a human papillomavirus (HPV) in a biological sample obtained from a subject in need thereof. In some embodiments, the methods comprise: (a) extracting DNA from the biological sample and amplifying the DNA by a fluorescent PCR using the kit as described herein; and (b) determining the presence or absence of DNA of one or more HPV subtype in the biological sample based on the results of the fluorescent PCR.

In some embodiments, the methods comprise detecting and/or identifying the presence or absence of DNA of at least one HPV subtypes in the biological sample. In some embodiments, the methods comprise detecting and/or identifying the presence or absence of DNA of 14 high-risk HPV subtypes in the biological sample through a single test tube, wherein the high-risk HPV subtypes are HPV16, HPV18, HPV31, HPV33, HPV35, HPV39, HPV45, HPV51, HPV52, HPV56, HPV58, HPV59, HPV66, and HPV68.

In some embodiments, the methods comprise detecting and/or identifying the presence or absence of DNA of 14 high-risk HPV subtypes in the biological sample through a single test tube, wherein the high-risk HPV subtypes are HPV16, HPV18, HPV31, HPV33, HPV35, HPV39, HPV45, HPV51, HPV52, HPV56, HPV58, HPV59, HPV66, and HPV68.

In some embodiments, the methods comprise detecting and/or identifying the presence or absence of DNA of 14 high-risk HPV subtypes and at least one low risk HPV subtype in the biological sample through two test tubes, wherein, high risk HPV subtypes are HPV16, HPV18, HPV31, HPV33, HPV35, HPV39, HPV45, HPV51, HPV52, HPV56, HPV58, HPV59, HPV66, and HPV68, and low-risk HPV subtypes are HPV6 and HPV11. The presence or absence of 7 high-risk HPV subtypes HPV16, HPV18, HPV35, HPV39, HPV51, HPV56, HPV59, HPV66 and HPV68 in the biological samples are detected and/or identified in one test tube, whereas the presence or absence of 5 high-risk HPV subtypes HPV31, HPV33, HPV45, HPV52 and HPV68, and the presence or absence of 2 low-risk HPV subtypes, HPV6 and/or HPV11, in the biological samples are detected and/or identified in the other test tube.

In some embodiments, the biological sample is a smear of the cervix, a fresh tissue sample, a fixed tissue sample, a cross-sectional specimen of a tissue sample, a urine sample, a sample comprising exfoliated cells, a peripheral blood sample, a penis swab, or other body fluid. In some embodiments, the sample is a urine sample.

The present disclosure further provides use of a combination of primer and probe or a kit as described herein for detecting and/or identifying the genotype of a human papillomavirus (HPV).

The present disclosure further provides methods for treating a condition associated with human papillomavirus (HPV) in a subject in need thereof. In some embodiments, the methods comprise: (1) detecting and/or identifying the genotype of a human papillomavirus (HPV) in a biological sample obtained from a subject in need thereof. In some embodiments, the step (1) comprises: (a) amplifying DNA extracted from the biological sample by a fluorescent PCR using a combination of primers and probes as described herein; and (b) determining the presence or absence of DNA of one or more HPV subtype in the biological sample based on the results of the fluorescent PCR. In some embodiments, the methods further comprise (2) treating the subject with a pharmaceutical composition and/or a medical procedure according to the results in step (1).

In some embodiments, the condition is a precancerous lesions caused by HPV. In some embodiments, the pharmaceutical composition comprises an antiviral agent.

The present disclosure further provides methods for vaccinating a human subject in need thereof. In some embodiments, the methods comprise (1) detecting and/or identifying the genotype of a human papillomavirus (HPV) in a biological sample obtained from the human subject in need thereof before and/or after the human subject is vaccinated. In some embodiments, the step (1) comprises (a) amplifying DNA extracted from the biological sample by a fluorescent PCR using a combination of primers and probes as described herein; and (b) determining the presence or absence of DNA of one or more HPV subtype in the biological sample based on the results of the fluorescent PCR. In some embodiments, the methods further comprise (2) vaccinating the subject with a composition targeting selected HPVs based on the results in step (1).

The present disclosure further provides methods for evaluating vaccination efficacy in a human subject in need thereof. In some embodiments, the methods comprise: (1) detecting and/or identifying the genotype of a human papillomavirus (HPV) in a biological sample obtained from the human subject in need thereof after, or before and after the human subject is vaccinated. In some embodiments, the step (1) comprises: (a) amplifying DNA extracted from the biological sample by a fluorescent PCR using a combination of primers and probes as described herein after, or before and after the human subject is vaccinated; and (b) determining the presence or absence of DNA of one or more HPV subtype in the biological sample based on the results of the fluorescent PCR after, or before and after the human subject is vaccinated. In some embodiments, the methods further comprise (2) vaccinating the subject with a composition targeting selected HPVs; and (3) determining vaccination efficacy based on the results in step (1).

The present disclosure further provides primers or pairs of primers related to HPVs. In some embodiments, the primers comprises a oligonucleotide sequence having at least 85%, 90%, 95%, or 100% identity to any one of SEQ ID NOs: 1-36. In some embodiments, the primers have less than 100, 90, 80, 70, 60, 50, 40, 30, or 20 nucleotides. In some embodiments, the pairs of primers comprise a forward primer and a reverse primer. In some embodiments, each primer in the pairs of primers have at least 85%, 90%, 95%, or 100% identity to any one of SEQ ID NOs: 1-36. In some embodiments, each primer in the pairs of primers have less than 100, 90, 80, 70, 60, 50, 40, 30, or 20 nucleotides. In some embodiments, the forward and reverse primers in the pairs of primers are selected from the groups consisting of: SEQ ID NOs: 1-2, 3-4, 5-6, 7-8, 9-10, 11-12, 13-14, 15-16, 17-18, 19-20, 21-22, 23-24, 25-26, 27-28, 29-30, 31-32, 33-34, 35-36, and any combination thereof.

The present disclosure further provides probes related to HPVs. In some embodiments, the probes comprise a fluorescent dye and an oligonucleotide. In some embodiments, the oligonucleotide comprises a sequence having at least 85%, 90%, 95%, or 100% identity to any one of SEQ ID NOs: 37-45. In some embodiments, the oligonucleotide has less than 100, 90, 80, 70, 60, 50, 40, 30, or 20 nucleotides.

In some embodiment, the fluorescent dye is attached to 5′ end of the probe. In some embodiments, the fluorescent dye is selected from the group consisting of FAM (fluorescein), TET, JOE, VIC, HEX, ROX, TAMRA, Cy3, cy3.5, Cy5, Cy5.5, OregonGreen™, CALRed™, Red640, Texas Red, LighterCycler®Cyan500, LighterCycler®, Red610, a biotin binding material, Alexa 647, Alexa 555, 5-(2-aminoethyl)amino-1-naphthalene sulfonic acid (EDANS), tetramethyl rhodamine (TMR), tetramethyl rhodamine isocyanate (TMRITC), fluorescein isocyanate (FITC), and χ-rhodamine.

In some embodiments, the probes further comprise comprising a fluorescent quencher. In some embodiments, the fluorescent quencher is attached to 3′ end of the probe. In some embodiments, the fluorescent quencher is selected from the group consisting of DDQ-I, DDQ-II, Dabcyl, Eclipse, Iowa Black FQ, Iowa Black RQ, BHQ-1, BHQ-2, BHQ-3, QSY-7, QSY-9, and QSY-21.

The present disclosure further provides kits for detecting and/or identifying the genotype of a human papillomavirus (HPV) in a biological sample. In some embodiments, the kits comprise one or more primers as described herein, and one or more probes as described herein. In some embodiments, the kits comprise at least 2, 4, 6, 8, 10, 12, 14, 16, 18, 20, 22, 24, 26, 28, 30, 32, 34, or 36 primers as described herein. In some embodiments, the kits comprise at least 2, 3, 4, 5, 6, 7, 8, or 9 probes as described herein. In some embodiments, the kits further comprise a lysis solution, magnetic nanoparticles, a protease, a first washing buffer, a second washing buffer, an elution buffer, or any combination thereof.

The present disclosure further provides methods for detecting and/or identifying the genotype of a human papillomavirus (HPV) in a urine sample obtained from a subject in need thereof. In some embodiments, the methods comprise: (a) obtaining DNA from the urine sample; (b) amplifying the DNA by a fluorescent PCR using one or more primers as described herein, and one or more probes as described herein; and (c) determining the presence or absence of DNA of one or more HPV subtype in the biological sample based on the results of the fluorescent PCR.

### BRIEF DESCRIPTION OF THE FIGURES

FIG. 1 depicts a phylogenetic tree showing similarity of L1 genes in 12 high-risk HPV subtypes.

FIG. 2 depicts alignment of L1 genes in 12 high-risk HPV subtypes and positons of probes (P1 to P4) that are designed for recognition of subgroups of these HPVs (HPV31, 33, 35, 58-P1; HPV39, 59, 68-P2; HPV45, 56, 66-P3; and HPV51, 52-P4).

FIG. 3A to FIG. 3Q depict the amplification curves using primers and probes specific to 14 high-risk HPVs and 2 low-risk HPVs, respectively (HPV16, HPV18, HPV31, HPV33, HPV35, HPV39, HPV45, HPV51, HPV52, HPV56, HPV58, HPV59, HPV66, and HPV68, HPV6 and HPV11) and the amplification curve of control gene β-actin in a multiplex PCR. Figures having more than one curves indicate that the same sample has been tested several times.

FIG. 4A to FIG. 4D depict the amplification curves using primers and probes specific to HPV16, HPV18, HPV33, and HPV6 in a multiplex PCR or in single-plex PCRs.

FIG. 5 depicts HPV16 gene amplification curves in multiplex PCRs using the preferred set of primers and probe (SEQ ID NO: 1, SEQ ID NO: 2, and SEQ ID NO: 37) or using a candidate set of primers and probe (SEQ ID NO: 46, SEQ ID NO: 47, and SEQ ID NO: 48).

### DETAILED DESCRIPTION OF THE INVENTION

Primers, Probes, and Multiplex PCR for HPV Subtype Detection and/or Genotyping

The present disclosure provides compositions and methods for HPV detection and genotyping.

Among HPV detection methods utilizing real-time PCR, E6/E7, E1 and L1 genes of HPV virus are commonly used as detection targets. Since HPV genotypes are distinguished by its L1 gene, the advantage of using L1 gene as the target to design primer and probe is that the primer and probe can have better genotype specificity. However, if the objective is to detect 14 high-risk HPV types in the same qPCR reaction, designing primers and probes for L1 genes that are both inclusive and specific is challenging, because these sequences are highly similar and cross-reactivity between primers and probes with unintended types need to be considered. By comparing and analyzing the L1 gene of 12 high-risk HPVs other than HPV16, and HPV18, we found four conservative area can be used to design probes (e.g., TaqMan probes). Comparing with the routine practice needing 12 specific probes, only four probes are needed to cover 12 high-risk HPVs other than HPV16 and HPV18. Together with optimized and combined type specific primers, we can realize the qPCR detection of 14 high-risk HPV types in the same reaction, or detection of 14 high-risk types and 2 additional low-risk types of HPV in a two-tube reaction.

In some embodiments, the present disclosure provides oligonucleotides that can be used as primers to amplify DNA of HPVs, and oligonucleotides that can be used as probes for detecting and/or identifying DNA of specific HPV genotypes.

In some embodiments, the HPV subtypes include at least 4, at least 5, at least 6, at least 7, at least 8, at least 9, at least 10, at least 11, at least 12, at least 13, or at least 14 of high-risk subtypes, such as HPV16, HPV18, HPV31, HPV33, HPV35, HPV39, HPV45, HPV51, HPV52, HPV56, HPV58, HPV59, HPV66, and HPV68, and one or two low-risk HPV subtypes such as HPV6 and HPV11.

In some embodiments, the primers and probes are specific to the L1 regions of HPV subtypes. In some embodiments, the primers and probes used in the HPV test are provided in Table 1 and Table 2.

TABLE 1
Primers for L1 gene in HPV subtypes

| SEQ ID | | |
|---|---|---|
| NO. | Name | Sequence |
| 1 | HPV16-F | GATTGTCCACCATTAGAGTTAATAAACAC |
| 2 | HPV16-R | GCCTGTAATGTAGTAAAGTCCATAGC |
| 3 | HPV18-F | ACAGGTATGCGTGCTTCACC |
| 4 | HPV18-R | TTTATTAAACAACTGGGAGTCAGAGG |
| 5 | HPV31-F | GGTGGTCCTGGCACTGATAATA |
| 6 | HPV31-R | CTCCAATAGGTGGTTTGCAACC |
| 7 | HPV33-F | TGGACAACCGGGTGCTGATAA |
| 8 | HPV33-R | CCCTGTTGGAGGCTTACATCC |
| 9 | HPV35-F | AATATGTTGGTAACTCTGGTACAGATAAC |
| 10 | HPV35-R | GTGTGCCTTTTCCCCAATGTTC |
| 11 | HPV39-F | GTGGTCGCAAGCAGGACATT |
| 12 | HPV39-R | CTGAATTTATTAGGATCGGGCAATGTC |
| 13 | HPV45-F | AGGTACATATGATCCTACTAAGTTTAAGC |
| 14 | HPV45-R | ACCTCTGCAGTTAAAGTAATAGTGC |
| 15 | HPV51-F | TCACGCATAGCAAATGGCAATG |
| 16 | HPV51-R | CCAGTGTTCCCCAATAGGTGG |
| 17 | HPV52-F | GGTAAACCTGGTATAGATAATAGGGAAT |
| 18 | HPV52-R | TCCTGAATTATTATTACAAGGGGTTCC |
| 19 | HPV56-F | GCTACAGAACAGTTAAGTAAATATGATGC |
| 20 | HPV56-R | ATATTATGTAAATATGCCATAACCTCTGC |
| 21 | HPV58-F | GCAGGGTCTGATAACAGGGAAT |
| 22 | HPV58-R | GCTCACCAGTGGGAGGTTTACA |
| 23 | HPV59-F | AAGGTGGTAATGGTAGACAGGATG |
| 24 | HPV59-R | CGTTGAGAGTTAGGATCATATACTGTGT |
| 25 | HPV66-F | ACCAGAAGTACCAACATGACTATTAATG |
| 26 | HPV66-R | AGGTTATTTTACAAAGTTGAAACACAAAC |
| 27 | HPV68a-F | CTATGCTGGTACATCTAGGTTATTAACTG |
| 28 | HPV68a-R | ATCAGGTAAGGTAACCCTAAACACTC |
| 29 | HPV68b-F | ATTACTATGCTGGTACATCTAGGTTATTAA |
| 30 | HPV68b-R | GACTAAATTTATTAGGATCAGGTAGGGAA |
| 31 | ACTB-F | AGGCATCCTCACCCTGAAGTAC |
| 32 | ACTB-R | ACACGCAGCTCATTGTAGAAGG |
| 33 | HPV6-F | GGGTAATCAACTGTTTGTTACTGTG |
| 34 | HPV6-R | CATGACGCATGTACTCTTTATAATCAG |
| 35 | HPV11-F | TGCATTACCTGATTCATCTCTGTTTG |
| 36 | HPV11-R | CATCATATTTGTTTAGCAATGGATGCC |

TABLE 2
Probes specific for L1 gene in HPV subtypes

| SEQ | | |
|---|---|---|
| ID | | |
| NO. | Name | Sequence |
| 37 | HPV16-P | TGACCACGACCTACCTCAACACCTACACAG |
| 38 | HPV18-P | TGTGTGTATTCTCCCTCTCCAAGTGGCTCT |
| 39 | HPV31, 33, | ATGGATTATAAACAAACACA |
| | 35, 58-P1 | |
| 40 | HPV39, 59, | TATTGATATGCAGACAC |
| | 68-P2 | |
| 41 | HPV45, 56, | CATGTGGAGGAATATGA |
| | 66-P3 | |
| 42 | HPV51, 52-P4 | CAGACTCAGTTATG |
| 43 | ACTB-P | CGAGCACGGCATCGTCACCAACTGG |
| 44 | HPV6-P | AGATACCACACGCAGTACCAACATGACATT |
| 45 | HPV11-P | CCACTACACAGCGTTTAGTATGGGCGTGC |

The oligonucleotides of the present disclosure, in particular those having the nucleotide sequences recited in SEQ ID NOs: 1 to 30 and 33 to 36, advantageously permit the extremely specific amplification of L1 genes of corresponding HPV subtypes in a biological sample that potentially comprises different human HPV subtypes.

The oligonucleotides of the present disclosure, in particular those having the nucleotide sequences recited in SEQ ID NOs: 37 to 42 and 44 to 45, specifically recognize L1 genes of corresponding HPV subtypes in a biological sample that potentially comprises different human HPV subtypes.

The oligonucleotides of the present disclosure, in particular those having the nucleotide sequences recited in SEQ ID NOs: 31 to 32 and 43, can specifically amplify and recognize a β-actin gene in a biological sample.

In some embodiments, also provided are oligonucleotides that are highly similar to a sequence of SEQ ID NOs: 1 to 45. In some embodiments, such oligonucleotides have at least 60%, 65%, 70%, 75%, 80%, 85%, 90%, 91%, 92%, 93%, 94%, 95%, 96%, 97%, 98%, 99%, or more identity to a sequence of SEQ ID NOs: 1 to 45. In some embodiments, also provided are oligonucleotides that hybridize to a sequence of SEQ ID NOs: 1 to 45 under a stringent hybridization condition. In some embodiments, also provided are oligonucleotides that are functional variants of a sequence of SEQ ID NOs: 1 to 45 under a stringent hybridization condition.

In some embodiments, provided are oligonucleotides that are partially or completely complementary to a sequence of SEQ ID NOs: 1 to 45.

In some embodiments, provided are oligonucleotides having one or more modifications compared to a sequence of SEQ ID NOs: 1 to 45. In some embodiments, the oligonucleotides are obtained through a) deletion of 1, 2, 3, 4, 5, 6, 7, 8, 9, 10 nucleotides in one of the nucleotide sequences recited in SEQ ID NOs: 1 to 45; b) addition of 1, 2, 3, 4, 5, 6, 7, 8, 9, 10 nucleotides in one of the nucleotide sequences recited in SEQ ID NOs: 1 to 45, and/or c) substitution of 1, 2, 3, 4, 5 nucleotides in one of the nucleotide sequences recited in SEQ ID NOs: 1 to 45. The modification can happen at the 5′ end and/or 3′ end of one of the nucleotide sequences recited in SEQ ID NOs: 1 to 45.

Examples of modified base moieties which can be used to modify nucleotides at any position on its structure include, but are not limited to: 5-fluorouracil, 5-bromouracil, 5-chlorouracil, 5-iodouracil, hypoxanthine, xanthine, acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N-6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, methoxyarninomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid, pseudouracil, queosine, 2-thiocytosine, 5-methyl-2-thiouracil, 2-thiouracil, 4-thiouracil, 5-methyluracil, uracil-5-oxyacetic acid methylester, uracil-S-oxyacetic acid, 5-methyl-2-thiouracil, 3-(3-amino-3-N-2-carboxypropyl) uracil, and 2,6-diaminopurine amongst others.

Examples of modified sugar moieties which may be used to modify nucleotides at any position on its structure include, but are not limited to: arabinose, 2-fluoroarabinose, xylose, and hexose, or a modified component of the phosphate backbone, such as phosphorothioate, a phosphorodithioate, a phosphoramidothioate, a phosphoramidate, a phosphordiamidate, a methylphosphonate, an alkyl phosphotriester, or a formacetal or analog thereof.

In some embodiments, an oligonucleotide in a sequence of SEQ ID NOs: 1-45 is replaced by a unnatural nucleotide, such as an artificial nucleic acid. Artificial nucleic acids include, but are not limited to, peptide nucleic acid (PNA), Morpholino, locked nucleic acid (LNA), glycol nucleic acid (GNA) and threose-nucleic acid (TNA). Each of these is distinguished from naturally occurring DNA or RNA by changes to the backbone of the molecule.

The present disclosure provides primer pairs to amplify a nucleic acid region of HPV subtypes. Each pair of primers comprises a forward primer and a reverse primer. In some embodiments, these primer pairs include:

a forward primer comprising, consisting of, or consisting essentially of the polynucleotide sequence of SEQ ID NO: 1, a reverse primer comprising the polynucleotide sequence of SEQ ID NO: 2, for amplifying a sequence in HPV16;
a forward primer comprising, consisting of, or consisting essentially of the polynucleotide sequence of SEQ ID NO: 3, a reverse primer comprising the polynucleotide sequence of SEQ ID NO: 4, for amplifying a sequence in HPV18;
a forward primer comprising, consisting of, or consisting essentially of the polynucleotide sequence of SEQ ID NO: 5, a reverse primer comprising the polynucleotide sequence of SEQ ID NO: 6, for amplifying a sequence in HPV31;
a forward primer comprising, consisting of, or consisting essentially of the polynucleotide sequence of SEQ ID NO: 7, a reverse primer comprising the polynucleotide sequence of SEQ ID NO: 8, for amplifying a sequence in HPV33;
a forward primer comprising, consisting of, or consisting essentially of the polynucleotide sequence of SEQ ID NO: 9, a reverse primer comprising the polynucleotide sequence of SEQ ID NO: 10, for amplifying a sequence in HPV35;
a forward primer comprising, consisting of, or consisting essentially of the polynucleotide sequence of SEQ ID NO: 11, a reverse primer comprising the polynucleotide sequence of SEQ ID NO: 12, for amplifying a sequence in HPV39;
a forward primer comprising, consisting of, or consisting essentially of the polynucleotide sequence of SEQ ID NO: 13, a reverse primer comprising the polynucleotide sequence of SEQ ID NO: 14, for amplifying a sequence in HPV45;
a forward primer comprising, consisting of, or consisting essentially of the polynucleotide sequence of SEQ ID NO: 15, a reverse primer comprising the polynucleotide sequence of SEQ ID NO: 16, for amplifying a sequence in HPV51;
a forward primer comprising, consisting of, or consisting essentially of the polynucleotide sequence of SEQ ID NO: 17, a reverse primer comprising the polynucleotide sequence of SEQ ID NO: 18, for amplifying a sequence in HPV52;
a forward primer comprising, consisting of, or consisting essentially of the polynucleotide sequence of SEQ ID NO: 19, a reverse primer comprising the polynucleotide sequence of SEQ ID NO: 20, for amplifying a sequence in HPV56;
a forward primer comprising, consisting of, or consisting essentially of the polynucleotide sequence of SEQ ID NO: 21, a reverse primer comprising the polynucleotide sequence of SEQ ID NO: 22, for amplifying a sequence in HPV58;
a forward primer comprising, consisting of, or consisting essentially of the polynucleotide sequence of SEQ ID NO: 23, a reverse primer comprising the polynucleotide sequence of SEQ ID NO: 24, for amplifying a sequence in HPV59;
a forward primer comprising, consisting of, or consisting essentially of the polynucleotide sequence of SEQ ID NO: 25, a reverse primer comprising the polynucleotide sequence of SEQ ID NO: 26, for amplifying a sequence in HPV66;
a forward primer comprising, consisting of, or consisting essentially of the polynucleotide sequence of SEQ ID NO: 27, a reverse primer comprising the polynucleotide sequence of SEQ ID NO: 28, for amplifying a sequence in HPV68a;
a forward primer comprising, consisting of, or consisting essentially of the polynucleotide sequence of SEQ ID NO: 29, a reverse primer comprising the polynucleotide sequence of SEQ ID NO: 30, for amplifying a sequence in HPV68b;
a forward primer comprising, consisting of, or consisting essentially of the polynucleotide sequence of SEQ ID NO: 33, a reverse primer comprising the polynucleotide sequence of SEQ ID NO: 34, for amplifying a sequence in HPV6; and a forward primer comprising, consisting of, or consisting essentially of the polynucleotide sequence of SEQ ID NO: 35, a reverse primer comprising the polynucleotide sequence of SEQ ID NO: 36, for amplifying a sequence in HPV11.

In some embodiments, the present disclosure provides a mixture comprising at least 2, 3, 4, 5, 6, 7, 8, 9, 10, 11, 12, 13, 14, 15, 16, or 17 pairs of primers as described herein. In some embodiments, the mixture comprises the primer pairs of (1) and (2). In some embodiments, the mixture comprises at least one primer pair selected from (1) and (2), and at least one primer pair selected from (3) to (17). In some embodiments, the mixture comprises at least one primer pair selected from (1) and (2), at least one primer pair selected from (3), (4), (5) and (11), at least one primer pair selected from (6), (12), (14) and (15), at least one primer pair selected from (7), (10), and (13), at least one primer pair selected from (8) and (9), the primer pair in (16), and/or the primer pair in (17), or any combinations thereof.

In some embodiments, the mixture comprises the primer pairs in (1) and (2), and the primer pairs in (3) to (15). In some embodiments, the mixture comprises the primer pairs in (1) and (2), the primer pairs in (3) to (15), and the primer pairs in (16) and/or (17).

In some embodiments, the mixture consists of, or consists essentially of the primer pairs in (1) and (2), and at least 1, 2, 3, 4, 5, 6, 7, 8, 9, 10, 11, 12 or 13 primer pairs in (3) to (15). In some embodiments, the mixture consists of, or consists essentially of the primer pairs in (1) and (2), at least 1, 2, 3, 4, 5, 6, 7, 8, 9, 10, 11, 12 or 13 primer pairs in (3) to (15), and the primer pairs in (16) and/or (17).

In some embodiments, the mixture further comprises one or more probes corresponding to one or more primer pairs in the mixture (e.g., a probe that matching a sequence amplified by a primer pair), as described herein.

The present disclosure provides probes that can specifically recognize sequences in HPVs. In some embodiments, the probes include:

(1) a probe comprising, consisting of, or consisting essentially of the sequence of SEQ ID NO: 37, for recognizing a sequence in HPV16;
(2) a probe comprising, consisting of, or consisting essentially of the sequence of SEQ ID NO: 38, for recognizing a sequence in HPV18;
(3) a probe comprising, consisting of, or consisting essentially of the sequence of SEQ ID NO: 39, for recognizing a sequence in HPV31, HPV33, HPV35, and/or HPV38;
(4) a probe comprising, consisting of, or consisting essentially of the sequence of SEQ ID NO: 40, for recognizing a sequence in HPV39, HPV59, and/or HPV68;
(5) a probe comprising, consisting of, or consisting essentially of the sequence of SEQ ID NO: 41, for recognizing a sequence in HPV45, HPV56, and/or HPV66;
(6) a probe comprising, consisting of, or consisting essentially of the sequence of SEQ ID NO: 42, for recognizing a sequence in HPV51 and/or HPV52;
(7) a probe comprising, consisting of, or consisting essentially of the sequence of SEQ ID NO: 44, for recognizing a sequence in HPV6;
(8) a probe comprising, consisting of, or consisting essentially of the sequence of SEQ ID NO: 45, for recognizing a sequence in HPV11.

In some embodiments, a probe of the present disclosure comprises a label at the 5′ and the probe.

In some embodiments, the label at the 5′ of a probe comprises a fluorescent dye, such as a fluorophore. As used herein, fluorophore is a fluorescent chemical compound that can re-emit light upon light excitation. Fluorophores typically contain several combined aromatic groups, or planar or cyclic molecules with several π bonds. Non-protein organic fluorophores include, but are not limited to, xanthene derivative (e.g., fluorescein, rhodamine, Oregon green, eosin, and Texas red); cyanine derivatives (e.g., cyanine, indocarbocyanine, oxacarbocyanine, thiacarbocyanine, and merocyanine), squaraine derivatives and ring-substituted squaraines (e.g., Seta, SeTau, and Square dyes), naphthalene derivatives (e.g., dansyl and prodan derivatives), coumarin derivatives; oxadiazole derivatives (e.g., pyridyloxazole, nitrobenzoxadiazole and benzoxadiazole); anthracene derivatives (e.g., anthraquinones, including DRAQS, DRAQ7 and CyTRAK Orange); pyrene derivatives (cascade blue, etc.), oxazine derivatives (e.g., Nile red, Nile blue, cresyl violet, oxazine 170, etc.; acridine derivatives (e.g., proflavin, acridine orange, acridine yellow, etc.); arylmethine derivatives (e.g., auramine, crystal violet, malachite green); tetrapyrrole derivatives (e.g., porphin, phthalocyanine, bilirubin). Particular examples include, but are not limited to, VIC, PET, Texas Red, Cy3, Cy5, FAM(6-Carboxyfluorescein), HEX (6-carboxy-2′,4,4′,5′,7,7′-hexachlorofluorescein), ROX (5(6)-Carboxy-X-rhodamine), JOE (6-carboxy-4′,5′-dichloro-21,71-dimethoxyfluorescein), TET (5′-tetrachloro-fluorescein phosphoramidite), NED (fluorescein benzoxanthene), TAMRA (6-carboxy-N,N,N,N-tetramethylrhodamine), FITC (fluorescein isothiocyanate). Examples of particular fluorophores that can be used in the probes disclosed herein are known to those of skill in the art and include those provided in U.S. Pat. No. 5,866,366 to Nazarenko et al., such as 4-acetamido-4′-isothiocyanatostilbene-2,2′disulfonic acid; acridine and derivatives such as acridine and acridine isothiocyanate, 5-(2′-aminoethyl)aminonaphthalene-1-sulfonic acid (EDANS), 4-amino-N-[3-vinylsulfonyl)phenyl]naphthalimide-3,5 disulfonate (Lucifer Yellow VS), N-(4-anilino-1-naphthyl)maleimide, anthranilamide; Brilliant Yellow; coumarin and derivatives such as coumarin, 7-amino-4-methylcoumarin (AMC, Coumarin 120), 7-amino-4-trifluoromethylcouluarin (Coumaran 151); cyanosine; 4′,6-diaminidino-2-phenylindole (DAPI); 5′,5″-dibromopyrogallol-sulfonephthalein (Bromopyrogallol Red); 7-diethylamino-3-(4′-isothiocyanatophenyl)-4-methylcoumarin; diethylenetriamine pentaacetate; 4,4′-diisothiocyanatodihydro-stilbene-2,2′-disulfonic acid; 4,4′-diisothiocyanatostilbene-2,2′-disulfonic acid; 5-[dimethylamino]naphthalene-1-sulfonyl chloride (DNS, dansyl chloride); 4-dimethylaminophenylazophenyl-4′-isothiocyanate (DABITC); eosin and derivatives such as eosin and eosin isothiocyanate; erythrosin and derivatives such as erythrosin B and erythrosin isothiocyanate; ethidium; fluorescein and derivatives such as 5-carboxyfluorescein (FAM), 5-(4,6-dichlorotriazin-2-yl)aminofluorescein (DTAF), 2′7′-dimethoxy-4′5′-dichloro-6-carboxyfluorescein (JOE), fluorescein, fluorescein isothiocyanate (FITC), QFITC (XRITC), -6-carboxy-fluorescein (HEX), and TET (Tetramethyl fluorescein); fluorescamine; IR144; IR1446; Malachite Green isothiocyanate; 4-methylumbelliferone; ortho cresolphthalein; nitrotyrosine; pararosanilin; Phenol Red; B-phycoerythrin; o-phthaldialdehyde; pyrene and derivatives such as pyrene, pyrene butyrate and succinimidyl 1-pyrene butyrate; Reactive Red 4 (CIBACRON™ Brilliant Red 3B-A); rhodamine and derivatives such as 6-carboxy-X-rhodamine (ROX), 6-carboxyrhodamine (R6G), lissamine rhodamine B sulfonyl chloride, rhodamine (Rhod), rhodamine B, rhodamine 123, rhodamine X isothiocyanate, N,N,N′,N′-tetramethyl-6-carboxyrhodamine (TAMRA), tetramethyl rhodamine, and tetramethyl rhodamine isothiocyanate (TRITC); sulforhodamine B; sulforhodamine 101 and sulfonyl chloride derivative of sulforhodamine 101 (Texas Red); riboflavin; rosolic acid and terbium chelate derivatives; LightCycler Red 640; Cy5.5; and Cy56-carboxyfluorescein; boron dipyrromethene difluoride (BODIPY); acridine; stilbene; 6-carboxy-X-rhodamine (ROX); Cy3; Cy3.5, Cy5, Cy5.5, VIC® (Applied Biosystems); LC Red 640; LC Red 705; OregonGreen™; CALRed™; Red640; and Yakima yellow; LighterCycler®Cyan500; LighterCycler®; Red610; Alexa 647; Alexa 555; 5-(2-aminoethyl)amino-1-naphthalene sulfonic acid (EDANS); tetramethyl rhodamine (TMR); tetramethyl rhodamine isocyanate (TMRITC), fluorescein isocyanate (FITC), χ-rhodamine, derivatives thereof, or any combination thereof, amongst others. More fluorescent dyes are described in U.S. Pat. Nos. 5,866,366, 6,818,431, 6,056,859, 9,140,688, 9,581,587, 6,165,765, 6,485,909, 8,158,358, 7,625,723, 7,560,236, 7,867,701, 9,150,912, 7,960,543, 6,555,383, 6,881,570, 8,198,026, 5,625,081, 8,445,291, 9,194,801, 8,835,110, 7,893,227, 9,243,289, 7,427,674, 9,512,493, US Patent Application Publication NOs: 20170152552, 20030170672, 20160281151, 20130084558, 20060281100, 20140234833, 20150072340, 20050089910, 20090081677, 2014002402220180171393, 20060188886, 20010018185, 20110151446, and WO/2000/017330A1, WO/2008/030071A1, WO/2013/049631A1, WO/2016/179090A1, WO/2016/123895A1, WO/2003/079022A1, each of which is herein incorporated by reference in its entirety.

In some embodiments, a probe of the present disclosure comprises a fluorescent donor and an acceptor fluorophore. As used herein, an acceptor fluorophore (e.g., a “fluorescent quencher”), is a fluorophore which absorbs energy from a donor fluorophore, for example in the range of about 400 to 900 nm. Acceptor fluorophores generally absorb light at a wavelength which is usually at least 10 nm higher (such as at least 20 nm higher) than the maximum absorbance wavelength of the donor fluorophore. Acceptor fluorophores have an excitation spectrum which overlaps with the emission of the donor fluorophore, such that energy emitted by the donor can excite the quencher. Any acceptor fluorophores known in the art can be utilized. In a particular example, an acceptor fluorophore is a dark quencher, such as Dabcyl, QSY7 (Molecular Probes), QSY9 (Molecular Probes), QSY21 (Molecular Probes), QSY33 (Molecular Probes), BLACK HOLE QUENCHERS™ (Glen Research, e.g., BHQ-1, BHQ-2, BHQ-3), ECLIPSE™ Dark Quencher (Epoch Biosciences), DDQ-I, DDQ-II, Dabcyl, Eclipse, or IOWA BLACK™ (Integrated DNA Technologies, e.g., Iowa Black FQ, Iowa Black RQ). More fluorescent quenchers are described in U.S. Pat. Nos. 9,957,546, 9,274,008, US Patent Publication NOs: 20140295422, 20090042205, 20160281182, 20180142284, 20140147929, and WO/2009/009615A1, WO/2016/160572A1, WO/2016/178953A1, WO/2018/229663A1, WO/2010/051544A2, WO/2013/152220A2, each of which is herein incorporated by reference in its entirety. A quencher can reduce or quench the emission of a donor fluorophore. In such an example, instead of detecting an increase in emission signal from the acceptor fluorophore when in sufficient proximity to the donor fluorophore (or detecting a decrease in emission signal from the acceptor fluorophore when a significant distance from the donor fluorophore), an increase in the emission signal from the donor fluorophore can be detected when the quencher is a significant distance from the donor fluorophore (or a decrease in emission signal from the donor fluorophore when in sufficient proximity to the quencher acceptor fluorophore).

In some embodiments, primers and probes of the present disclosure are based on fluorescence resonance energy transfer (FRET). Examples of oligonucleotides using FRET that can be used to detect amplicons include linear oligoprobes, such as HybProbes, 5′ nuclease oligoprobes, such as TAQMAN® probes, hairpin oligoprobes, such as molecular beacons, scorpion primers and UniPrimers, minor groove binding probes, and self-fluorescing amplicons, such as sunrise primers.

In some embodiments, primers and/or probes of the present disclosure are labeled by other functional entities, such as biotin, haptenes, antigens, chemical groups, radioactive substances, enzymatic markers, etc. The detection of a marked amplification product may be accomplished, for example, using fluorescence methods, chemoluminescence methods, densitometry methods, photometry methods, precipitation reactions, enzymatic reactions including enzymatic reinforcement reactions, SPR (“surface plamon resonance”) methods, ellipsometry methods, measurement of the index of refraction, measurement of reflectance, and similar methods.

In some embodiments, primers and probes of the present disclosure are used in a multiplex PCR system for amplifying sequences of at least 2, 3, 4, 5, 6, 7, 8, 9, 10, 11, 12, 13, 14, 15, 16 or 17 different HPV subtypes. In some embodiments, the multiplex PCR system further comprises oligonucleotides for amplifying and detecting a reference control oligonucleotide sequence.

In some embodiments, the multiplex PCR system can be used to detect and/or genotyping HPV subtypes in a biological sample. In some embodiments, the multiplex PCR system is established in a single test tube for detecting and/or genotyping at least 2, 3, 4, 5, 6, 7, 8, 9, 10, 11, 12, 13, 14, 15, 16 or 17 different HPV subtypes. In some embodiments, the HPV subtypes are selected from the group consisting of HPV16, HPV18, HPV31, HPV33, HPV35, HPV39, HPV45, HPV51, HPV52, HPV56, HPV58, HPV59, HPV66, and HPV68, HPV6 and HPV11. In some embodiments, probes in the multiplex PCR system are marked with different labels. In some embodiments, probes in the multiplex PCR system are marked with different fluorescent dyes. In some embodiments, the probe for HPV16 and/or HPV18 is labeled with a fluorescent dye that is different from the fluorescent dyes labeling the probes for other 12 high-risk HPVs (i.e., HPV31, HPV33, HPV35, HPV39, HPV45, HPV51, HPV52, HPV56, HPV58, HPV59, HPV66 and HPV68). In some embodiments, the probes for the 12 high-risk HPVs other than HPV16, HPV18 are labeled with the same fluorescent dye.

In some embodiments, the multiplex PCR system comprises primers and probes for detecting and/or genotyping high-risk HPV16 and/or HPV18. In some embodiments, the probes for detecting HPV16 and HPV18 are HPV16-P and HPV18-P as described herein, respectively. In some embodiments, HPV16-P is labeled by a first dye, and HPV18-P is labeled by a second dye.

In some embodiments, the multiplex PCR system further comprises primers and probes for detecting and/or genotyping at least 1, 2, 3, 4, 5, 6, 7, 8, 9, 10, 11, or 12 other high-risk HPV subtypes (i.e., HPV31, HPV33, HPV35, HPV39, HPV45, HPV51, HPV52, HPV56, HPV58, HPV59, HPV66, and HPV68). In some embodiments, the probe for detecting and/or genotyping HPV31, HPV33, HPV35, and/or HPV58 is HPV31, 33, 35, 58-P1 (SEQ ID NO: 39). In some embodiments, the probe for detecting and/or genotyping HPV39, HPV59, and/or HPV68 is HPV39, 59, 68-P2 (SEQ ID NO: 40). In some embodiments, the probe for detecting and/or genotyping HPV45, HPV56, and/or HPV66 is HPV45, 56, 66-P3 (SEQ ID NO: 41). In some embodiments, the probe for detecting and/or genotyping HPV51 and/or HPV52 is HPV51, 52-P4 (SEQ ID NO: 42). In some embodiments, the probe for detecting and/or genotyping HPV6 is HPV6-P (SEQ ID NO: 44). In some embodiments, the probe for detecting and/or genotyping HPV11 is HPV11-P (SEQ ID NO: 45). In some embodiments, the probe HPV31, 33, 35, 58-P1 comprises a third dye. In some embodiments, the probe HPV39, 59, 68-P2 comprises a fourth dye. In some embodiments, the probe HPV45, 56, 66-P3 comprises a fifth dye. In some embodiments, the probe HPV51, 52-P4 comprises a sixth dye. In some embodiments, the probe HPV6-P comprises a seventh dye. In some embodiments, the probe HPV11-P comprises an eighth dye. In some embodiments, the first dye and the second dye are different. In some embodiments, the third dye to the sixth dye are the same, but different from the dye in HPV16-P and HPV18-P. In some embodiments, the seventh dye in HPV6-P is different from the dye in HPV11-P, and different from the dyes in HPV16-P, HPV18-P, HPV31, 33, 35, 58-P1, HPV39, 59, 68-P2, HPV45, 56, 66-P3, and HPV51, 52-P4. In some embodiments, the eighth dye in HPV11-P is different from the dye in HPV6-P, and different from the dyes in HPV16-P, HPV18-P, HPV31, 33, 35, 58-P1, HPV39, 59, 68-P2, HPV45, 56, 66-P3, and HPV51, 52-P4.

In some embodiments, the multiplex PCR system comprises at least one pair of primers and at least one probe for the detection of at least one reference control gene. In some embodiments, the reference control gene is a gene in a subject whose activity would not be affected by the presence or absence of any HPV. In some embodiments, the reference control genes include, but are not limited to β-globin (HBB), telomerase (TERT), glyceraldehyde-3-phosphate dehydrogenase (GAPDH), albumin (ALB), β-actin (ACTB) and T cell receptor y (TRG). In some embodiments, the reference control gene is an actin gene in the subject, such as β-actin (ACTB). The present disclosure further provides primers for amplifying ACTB in a biological sample, such as SEQ ID NOs: 31 to 32. The present disclosure further provides a probe for detecting ACTB in a biological sample, such as SEQ ID NO. 43. In some embodiments, the probe for ACTB comprises a ninth dye, which is different from any of the dyes used in the probes for any of the HPV subtypes.

As a non-limiting example, provided is a multiplex PCR system for detecting and/or genotyping 14 high-risk HPV subtypes (i.e., HPV16, HPV 18, HPV31, HPV33, HPV35, HPV39, HPV45, HPV51, HPV52, HPV56, HPV58, HPV59, HPV66, and HPV68). In some embodiments, the multiplex PCR system can further detect and/or genotype 2 low-risk HPV subtypes (HPV6, HPV11) in addition to the 14 high-risk HPVs. In some embodiments, the multiplex PCR system is established in a single test tube. In some embodiments, the multiplex PCR system is established in two test tubes. In some embodiments, whether a single-test-tube system or a two-test-tube system is chosen, depends on the numbers of fluorescence detection channels in a qPCR instrument.

For example, in some embodiments, when the qPCR instrument has at least 6 detection channels (e.g., can recognize at least 6 different fluorescent dyes), the single-test-tube system can be chosen, in which:

(i) the probe for HPV16 has a first dye, such as Cy5 fluorescent dye attached to its 5′ end, and a quencher, such as BHQ-2 fluorescent quencher attached to its 3′ end;
(ii) the probe for HPV18 has a second dye, such as FAM fluorescent dye attached to its 5′ end, and a quencher, such as BHQ-1 fluorescent quencher attached to its 3′ end;
(iii) the probes for HPV31, HPV33, HPV45, HPV52, and HPV58 (e.g., HPV31, 33, 35, 58-P1, HPV45, 56, 66-P3, and HPV51, 52-P4) are used, and all have the same dye (e.g., a third dye).
(iv) the probes for HPV35, HPV39, HPV51, HPV56, HPV59, HPV66 and HPV68 (e.g., HPV31, 33, 35, 58-P1, HPV39, 59, 68-P2, HPV45, 56, 66-P3, and HPV51, 52-P4) all have the same dye (e.g., a fourth dye), such as a VIC fluorescent dye attached to their 5′ ends, and a quencher, such as MGBNFQ fluorescent quencher attached to their 3′ end;
(v) the probe for the reference control gene has a fifth dye, such as ROX fluorescent dye attached to its 5′ end, and a quencher, such as BHQ-2 fluorescent quencher attached to its 3′ end; and
(vi) the probes for HPV6 and HPV11 have the same dye as a sixth dye, which is different from the dyes in (i) to (v).

For another example, in some embodiments, when the qPCR instrument has less than 6 but at least 4 detection channels (e.g., can recognize at least 4, but less than 6 different fluorescent dyes), a two-test-tube system can be chosen, which uses a first test tube and a second test tube. Each test tube at least comprises a probe for the detection of a reference control gene that has a first dye, such as ROX fluorescent dye attached to its 5′ end, and a quencher, such as BHQ-2 fluorescent quencher attached to its 3′ end. The following probes can be distributed into the two test tubes, as long as the two test tubes together cover all 14 high-risk HPV subtypes and the 2 low-risk HPV subtypes, in addition to the reference control gene, without causing conflict among the fluorescence detection channels. For example, in some embodiments the following strategy can be taken:

(i) in the first test tube, a probe for HPV16 having a second dye, such as Cy5 fluorescent dye attached to its 5′ end, and a quencher, such as BHQ-2 fluorescent quencher attached to its 3′ end;
(ii) in the first test tube, a probe for HPV18 having a third dye, such as FAM fluorescent dye attached to its 5′ end, and a quencher, such as BHQ-1 fluorescent quencher attached to its 3′ end;
(iii) in the first test tube, the probes for HPV31, HPV33, HPV45, HPV52, and HPV58(e.g., HPV31, 33, 35, 58-P1, HPV45, 56, 66-P3, and HPV51, 52-P4) all have the same dye (e.g., a fourth dye), such as a VIC fluorescent dye attached to their 5′ ends, and a quencher, such as MGBNFQ fluorescent quencher attached to their 3′ end;
(iv) in the second test tube, the probes for HPV35, HPV39, HPV51, HPV56, HPV59, HPV66, and HPV68 (e.g., HPV31, 33, 35, 58-P1, HPV39, 59, 68-P2, HPV45, 56, 66-P3, and HPV51, 52-P4) all have the same dye (e.g., a second dye), such as a VIC fluorescent dye attached to their 5′ ends, and a quencher, such as MGBNFQ fluorescent quencher attached to their 3′ end; and
(v) in the second test tube, the probes for HPV6 and HPV11 have the same dye as a third dye, such as HEX fluorescent dye attached to its 5′ end, and a quencher, such as BHQ-1 fluorescent quencher attached to its 3′ end.

In some embodiments, the probe for HPV16 is labeled with CY5 at its 5′ end, and with BHQ-2 at its 3′ end; the probe for HPV18 is labeled with FAM at its 5′ end, and with BHQ-1 at its 3′ end; the probes for other 12 high-risk HPVs (e.g., HPV31, 33, 35, 58-P1, HPV39, 59, 68-P2, HPV45, 56, 66-P3, and HPV51, 52-P4) are labeled with VIC at their 5′ ends, and with MGBNFQ at their 3′ ends; the probes for HPV6 and HPV11 are labeled with FAM at their 5′ ends, and with BHQ-1 at their 3′ ends; and the probe for the control gene (e.g., β-actin) is labeled with ROX at its 5′ end, and with BHQ-1 at its 3′ end.

### Methods Using the Primers and Probes

The present disclosure also provides methods for amplifying DNA of HPVs, and methods for detecting and/or genotyping HPV subtypes, using the primers and probes as described herein.

In some embodiments, the amplification products using the primers of the present disclosure can be used to detect the presence or absence of a given HPV subtype in a biological sample. The presence of an amplification product using one or more particular pairs of primers and probes described herein indicates the presence of one or more corresponding HPV subtypes in the biological sample being tested. The absence of an amplification product indicates that one or more corresponding HPV subtypes are not in the biological sample being tested.

In some embodiments, a qPCR is used for determining the presence or absence of a given HPV subtype. In some embodiments, a positive reaction is detected by accumulation of a fluorescent signal. The cycle threshold (Ct) is defined as the number of cycles required for the fluorescent signal to cross the threshold (e.g., exceeding the background level). In some embodiments, the threshold is automatically determined by the software of the qPCR instrument or other suitable methods. In some embodiments, the threshold is set just above (e.g., about 0.01%, 0.1%, 1%, 5%, or 10% higher) the terminal fluorescent value in the negative control sample. In some embodiments, when the Ct value associated with a HPV subtype amplification in a test sample is no more than (1 about 35, 34, 33, 32, 31, 30, or less, the sample is determined as containing the HPV subtype (positive result), otherwise the sample is determined as not containing the HPV subtype (negative result). For the reference control gene amplification, when the Ct value associated with a control gene amplification in the sample is no more than (≤) about 35, 34, 33, 32, 31, 30, 29 or less, the reference control gene amplification is determined to be positive, otherwise the reference control gene amplification is determined to be negative. When the reference control gene amplification is determined to be negative, and HPV gene amplification results are also negative, the test result is invalidated.

Methods using primers and probes of the present disclosure provide unexpected sensitivity and specificity of HPV detection and/or HPV genotyping. In some embodiments, the primers and probes of the present disclosure provide very high sensitivity and specificity of HPV detection and/or HPV genotyping, particularly when the sample is a urine sample.

As used herein, the term “sensitivity” refers to the rate when samples actually containing HPV in a given population are correctly diagnosed as having HPV by using a method of the present invention. In some embodiments, the sensitivity of a detection/genotyping method of the present disclosure is at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or more. In some embodiments, the size of the population is at least about 50, 100, 200, 300, 400, 500, 600, 700, 800, 900, 1000, 2000, 3000, 4000, 5000, 6000, 7000, 8000, 9000, 10,000, or more.

As used herein, the term “specificity” refers to the rate when samples actually not containing HPV in a given population are correctly diagnosed as not having HPV. In some embodiments, the specificity of a detection/genotyping method of the present disclosure is at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or more. In some embodiments, the size of the population is at least about 50, 100, 200, 300, 400, 500, 600, 700, 800, 900, 1000, 2000, 3000, 4000, 5000, 6000, 7000, 8000, 9000, 10,000, or more.

Methods using primers and probes of the present disclosure are capable of detecting the presence of HPV DNA in a biological sample when the copy number of the HPV DNA in the sample is low. For example, the methods described herein can identify the presence of a HPV DNA when at least 1, 2, 3, 4, 5, 6, 7, 8, 9, 10, 15, 20 or more copies of the DNA are among 10⁵ DNA molecules in a sample. In some embodiments, for a multiplex PCR system descried herein, at least 1, 2, 3, 4, 5, 6, 7, 8, 9, 10, 11, 12, 13, or 14 of the high-risk HPV can be detected when at least 1, 2, 3, 4, 5, 6, 7, 8, 9, 10, 15, 20 or more copies of DNA derived from each HPV subtype are among 10⁵ DNA molecules in the sample.

Detection/genotyping methods of the present disclosure using a urine sample also provide results that are highly in agreement with results obtaining using a sample collected from the same subject through an invasive procedure, such as a cervical sample (e.g., cervical scraper), a vaginal sample (e.g., vaginal swab) or a urethral sample (e.g., a urethral swab). In some embodiments, the matching rate of using a urine sample and a sample obtained through the invasive procedure is at least about 89%, 90%, 91%, 92%, 93%, 94%, 95%, 96%, 97%, 98%, 99%, or more.

The present disclosure also provides methods for identifying/screening for a subject that carries HPV, using the primers and probes as described herein. In some embodiments, the methods comprise determining the presence or absence of one or more HPV subtypes in a biological sample collected from the subjects by amplifying DNA in the biological sample using the a pair of primers of the present disclosure. In some embodiments, the methods comprise hybridizing amplified DNA in the biological sample with a probe of the present disclosure. In some embodiments, the subject is a human, such as a female human subject or a male human subject. In some embodiments, the biological sample is one collected from the genitourinary system of the human subject. The human subject can be any one, such as those who have been suspected to have HPV infection, or those who are at risk of having HPV infection. In some embodiments, the human subject has one or more symptoms related to a condition associated with HPV, such as genital warts, common warts, plantar warts, flat warts, lesions, inflammation, bleeding, genital lumps and bumps, and genital itching, etc. In some embodiments, the human subject does not have any symptoms.

The present disclosure also provides methods for identifying for a subject that has risk of having pre-cancerous/premalignant cells (e.g., abnormal cells that could turn into cancerous cells but are not invasive themselves), and/or cancerous cells. In some embodiments, the pre-cancerous cells and/or the cancerous cells are caused by HPV infection, either by one or more high-risk HPV subtypes, and/or by one or more low-risk HPV subtypes, or mixture thereof. HPV related cancers include, but are not limited to, cervical cancer, vaginal cancer, vulvar cancer, penile cancer, anal cancer, and head and neck cancer (e.g., mouth and throat cancers). In some embodiments, the methods comprises detecting the presence or absence of one or more HPV subtypes in a biological sample collected from a subject being tested. In some embodiments, the presence of one or more HPV subtypes in the biological sample indicates that the subject is at risk of having pre-cancerous/premalignant cells.

The present disclosure also provides methods for providing direction to HPV vaccination in a subject in need thereof. In some embodiments, the methods comprise detecting and/or identifying HPV subtypes in a biological sample collected from a subject in need thereof. In some embodiments, based on the HPV test result, a suitable HPV vaccine is selected for the subject. For example, in some embodiments, the HPV vaccine should at least target one or more HPV subtypes identified in the biological sample of the subject.

The present disclosure also provides methods for evaluating HPV vaccination efficacy in a subject in need thereof. In some embodiments, the method comprises detecting and/or identifying HPV subtypes in a biological sample collected from a subject in need thereof before and/or after the subject receives HPV vaccination. In some embodiments, the HPV test result is used to determine the presence and/or level of one or more HPV subtypes in the biological sample before and/or after the vaccination, which in turn indicates the efficacy of the HPV vaccination. In some embodiments, the HPV vaccination can be either enhanced, repeated, paused, terminated, and/or substituted based on the HPV test result.

In some embodiments, methods described herein comprise conducting a PCR. In some embodiments, the PCR is a real-time PCR. In some embodiments, the real-time PCR is a quantitative or semi-quantitative PCR. The information obtained from the PCR, such as an amplification curve, can be used to determine the presence of a target nucleic acid (such as a HPV gene) and/or quantitate the initial amounts of a target nucleic acid sequence. In some examples, the amount of amplified target nucleic acid (such as a HPV gene) is detected using a labeled probe, such as a probe labeled with a fluorophore, for example a TAQMAN® probe. In this example, the increase in fluorescence emission is measured in real time, during the course of the real time PCR. This increase in fluorescence emission is directly related to the increase in target nucleic acid amplification (such as HPV gene amplification).

The present disclosure also provides methods for treating or preventing HPV infection and/or HPV related conditions (e.g., precancer or cancer) in a subject in need thereof. In some embodiments, the methods comprise detecting and/or genotyping HPV subtypes in a biological sample collected from the subject in need thereof, and treating the subject based on the detection/genotyping results. In some embodiments, the treatment include, but are not limited to, vaccination, surgery, chemotherapy, radiation therapy, immunotherapy, palliative care, and exercise, etc. As used herein the phrase “treatment regimen” refers to a treatment plan that specifies the type of treatment, dosage, schedule and/or duration of a treatment provided to a subject in need thereof (e.g., a subject diagnosed with a pathology). The selected treatment regimen can be an aggressive one which is expected to result in the best clinical outcome (e.g., complete cure of the pathology) or a more moderate one which may relieve symptoms of the pathology yet results in incomplete cure of the pathology. It will be appreciated that in certain cases the treatment regimen may be associated with some discomfort to the subject or adverse side effects (e.g., damage to healthy cells or tissue). The type of treatment can include a surgical intervention (e.g., removal of lesion, diseased cells, tissue, or organ), a cell replacement therapy, an administration of a therapeutic drug (e.g., receptor agonists, antagonists, hormones, chemotherapy agents) in a local or a systemic mode, an exposure to radiation therapy using an external source (e.g., external beam) and/or an internal source (e.g., brachytherapy) and/or any combination thereof. The dosage, schedule and duration of treatment can vary, depending on the severity of pathology and the selected type of treatment, and those of skills in the art are capable of adjusting the type of treatment with the dosage, schedule and duration of treatment.

In some embodiments, one or more probes and/or primers of the present disclosure are used in other HPV detection methods. Such methods include, but are not limited to, DNA chip, microarray, hybridization, and/or droplet microfluidic PCR technologies.

In some embodiments, in any of the method described herein, a biological sample contains urine collected from the subject being in need thereof. In some embodiments, the urine sample is been processed as described herein to release DNA in the exfoliated cells and/or potential virus in the urine sample. Methods described herein are suitable for a HPV DNA sample obtained by any known method in the field, such as those available virus DNA extraction methods, including but not limited to boiling, phenol chloroform, magnetic bead or other commercial separation methods. In some embodiments, reagents and methods for extracting DNA from a urine sample are those described in the present disclosure.

In some embodiments, in any of the method described herein, a biological sample include, but is not limited to, blood, sweat, tears, urine, saliva, semen, serum, plasma, cerebrospinal fluid (CSF), feces, vaginal fluid or tissue, sputum, nasopharyngeal aspirate or swab, lacrimal fluid, mucous, or epithelial swab (buccal swab), tissues, organs, bones, teeth, or tumors, among others, which is collected from the subject being in need thereof. In some embodiments, the biological sample is been processed as described herein to release DNA in the exfoliated cells and/or potential virus in the urine sample. Methods described herein are suitable for a HPV DNA sample obtained by any known method in the field, such as those available virus DNA extraction methods, including but not limited to boiling, phenol chloroform, magnetic bead or other commercial separation methods. In some embodiments, reagents and methods for extracting DNA from a urine sample are those described in the present disclosure.

### Compositions and Methods for DNA Extraction

The present disclosure also provides compositions and methods for extracting DNA from a biological sample collected from a subject. In some embodiments, the biological sample is collected from a mammalian subject, such as a human. In some embodiments, the biological sample is a urine sample. Non-limiting examples of biological samples include, blood, sweat, tears, urine, saliva, semen, serum, plasma, cerebrospinal fluid (CSF), feces, vaginal fluid or tissue, sputum, nasopharyngeal aspirate or swab, lacrimal fluid, mucous, or epithelial swab (buccal swab), tissues, organs, bones, teeth, or tumors, among others.

Compositions and methods of the present disclosure give a simple and cost-efficient way to extract DNA from a biological sample, such as a urine sample. Particularly, compositions and methods of the present disclosure enable simultaneously extracting DNA from exfoliated cells in the biological sample, and DNA from one or more pathogen in the sample. For example, in some embodiments, the DNA extraction compositions and methods of the present disclosure can extract DNA in a urine sample more effectively. In addition, compositions and methods of the present disclosure make it possible to conduct automated DNA extraction, thus reducing labor intensity whiling increasing the overall throughput.

In some embodiments, the present disclosure provides reagents for DNA extraction from a biological sample. In some embodiments, the biological sample is a urine sample. In some embodiments, the reagents comprise magnetic particles. In some embodiments, the reagents comprise a protease. In some embodiments, the reagents further comprise a lysis solution. In some embodiments, the reagents further comprise a first washing buffer. In some embodiments, the reagents further comprise a second washing buffer. In some embodiments, the reagents comprise further comprise an elution buffer. In some embodiments, said reagents can be either provided as a kit, or be provided separately before use.

In some embodiments, the magnetic particles and the protease are used to pretreat a urine sample and get it ready for DNA extraction.

In some embodiments, the lysis solution, the first washing buffer, the second washing buffer, and the elution buffer are used to extract DNA from the pretreated urine sample.

In some embodiments, DNA extraction of the present disclosure is based on magnetic particles, such as magnetic nanoparticles (e.g., magnetic nano beads).

In some embodiments, the magnetic particles have a magnetic core, protected by a coating. The coating prevents irreversible aggregation of the magnetic particles and allows functionalization by the attachment of ligands for adsorption of DNA. In some embodiments, magnetic particles are incubated in the sample for as long as necessary to achieve optimal adsorption. In some embodiments, the magnetic particles contain iron oxide, such as Fe₃O₄ or Fe₂O₃. In some embodiments, the iron oxide material is processed into magnetic ‘pigment’ by reducing its size to few nanometers, then the magnetic ‘pigment’ can be encapsulated in non-magnetic matrices such as silica, polyvinyl alcohol (PVA), dextran, agarose, sepharose, and polystyrene, which can be biofunctionalized and used for life science applications.

In some embodiments, the magnetic particles have a core-shell structure. In some embodiments, the magnetic particles have an embedded structure.

For a core-shell structure, the magnetic particles are composed of a single superparamagnetic core with a polymer or silica surface coating, such as a magnetic core surrounded with a SiO₂ shell. In some other embodiments, the magnetic particles are composed of a polystyrene or polyvinyl alcohol (PVA) core surrounded by superparamagnetic particles and protected by a surface coating. In some embodiments, the magnetic particles have multiple layers of superparamagnetic particles alternating with encapsulation material.

For an embedded structure, superparamagnetic beads can be composed of a monodisperse matrix such as polystyrene, agarose or sepharose, which are impregnated with multiple iron-oxide nanoparticles (“magnetic pigment”). These beads are typically hundreds of nanometers in diameter and are sealed with a material that prevents loss of the magnetic pigment.

Non-limiting examples of magnetic particles for DNA extraction can be found in U.S. Pat. Nos. 6,514,688, 6,673,631, 6,027,945, 8,710,211, 6,033,878, 6,368,800, 8,324,372, 8,729,252, U.S. Application Publication NOs: 20030087286, 20150141258, 20160102305, 20130096292, 20020086326, 20050287583, 20100009351, 20110171640, 20110008797, 20180195035, 20080132694, 20040002594, 20090131650, 20160369263, 20140288398, 20030224366, and WO/2001/037291A1, WO/2001/045522A1, WO/1998/031840A1, WO/2005/021748A1, WO/2017/051939A1, WO/2017/137192A1, WO/2010/005444A1, WO/1992/008805A1, WO/2013/164319A1, WO/2015/126340A1, WO/2017/156336A1, WO/2009/102632A3, WO/2009/102632A2, WO/2009/012185A1, WO/2009/012185A9, WO/2009/115335A1, WO/2015/120445A1, WO/2015/123433A2, WO/2007/050327A2, WO/2007/050327A3, and WO/2013/028548A2, each of which is herein incorporated by reference in its entirety for all purposes.

In some embodiments, the magnetic particles are hydroxyl magnetic beads, coated by silica.

In some embodiments, the magnetic particles are magnetic beads having an average diameter of about 50 nm, 60 nm, 70 nm, 80 nm, 90 nm, 100 nm, 150 nm, 200 nm, 250 nm, 300 nm, 350 nm, 400 nm, 450 nm, 500 nm, 550 nm, 600 nm, 650 nm, 700 nm, 750 nm, 800 nm, 850 nm, 900 nm, 950 nm, 1000 nm, or more.

Also provided is a solution containing the magnetic particles. The concentration of the magnetic particles in the solution be can predetermined as needed. In some embodiments, the concentration is about 5 mg/ml to about 100 mg/ml, about 100 mg/ml to 200 mg/ml, about 200 mg/ml to 300 mg/ml, about 300 mg/ml to 400 mg/ml, about 400 mg/ml to 500 mg/ml, or more. In some embodiments, the concentration is about 10 mg/ml, about 20 mg/ml, about 30 mg/ml, about 40 mg/ml, about 50 mg/ml, about 60 mg/ml, about 70 mg/ml, about 80 mg/ml, about 90 mg/ml, about 100 mg/ml, about 200 mg/ml, about 300 mg/ml, about 400 mg/ml, about 500 mg/ml, or more.

In some embodiments, the solution containing the magnetic particles is mixed with a sample containing DNA. In some embodiments, the final concertation of the magnetic particles after mixed with the sample is predetermined, based on potential or actual quantity of DNA in the sample. In some embodiments, the final working concentration of the magnetic particles after being mixed with the sample containing DNA is about 0.01 to 0.5 mg/ml. In some embodiments, the final working concentration is about 0.01 mg/ml, 0.02 mg/ml, 0.03 mg/ml, 0.04 mg/ml, 0.05 mg/ml, 0.06 mg/ml, 0.07 mg/ml, 0.08 mg/ml, 0.09 mg/ml, 0.1 mg/ml, 0.15 mg/ml, 0.2 mg/ml, 0.25 mg/ml, 0.3 mg/ml, 0.35 mg/ml, 0.4 mg/ml, 0.45 mg/ml, 0.5 mg/ml, or more.

In some embodiments, after the magnetic particles is mixed with a sample containing DNA are mixed, the mixture is shaken for a predetermined time. In some embodiments, optionally the mixture is set still for a certain period of time after being mixed. The mixture is then centrifuged at a predetermined speed to precipitate the magnetic particles. In some embodiments, the supernatant is removed and the precipitated magnetic particles is processed further for DNA extraction.

In some embodiments, the precipitated magnetic particles are processed by a protease. In some embodiments, the protease is a broad-spectrum protease. In some embodiments, the protease is a serine protease, a cysteine protease, a threonine protease, an aspartic protease, a glutamic protease, a metalloprotease, an asparagine peptide lyase.

In some embodiments, the serine protease is protease K (EC 3.4.21.64, proteinase K, endopeptidase K, Tritirachium alkaline proteinase, Tritirachium album serine proteinase, Tritirachium album proteinase K). In some embodiments, the term protease K also include any functional variants of a natural protease K.

Also provided is a solution containing a protease, such as protease K. The concentration of the protease in the solution be can predetermined as needed. In some embodiments, the concentration is about 1 mg/ml to about 100 mg/ml. In some embodiments, the concentration is about 1 mg/ml, about 2 mg/ml, about 3 mg/ml, about 4 mg/ml, about 5 mg/ml, about 6 mg/ml, about 7 mg/ml, about 8 mg/ml, about 9 mg/ml, about 10 mg/ml, about 11 mg/ml, about 12 mg/ml, about 13 mg/ml, about 14 mg/ml, about 15 mg/ml, about 16 mg/ml, about 17 mg/ml, about 18 mg/ml, about 19 mg/ml, about 20 mg/ml, about 30 mg/ml, about 40 mg/ml, about 50 mg/ml, about 60 mg/ml, about 70 mg/ml, about 80 mg/ml, about 90 mg/ml, about 100 mg/ml, or more.

In some embodiments, the precipitated magnetic particles are mixed with a solution comprising a protease, such as protease K. In some embodiments, the final concertation of the protease after mixed is predetermined. In some embodiments, the final working concentration of the protease after being mixed with the precipitated magnetic particle is about 5 to 500 μg/ml. In some embodiments, the final working concentration is about 5 μg/ml, 6 μg/ml, 7 μg/ml, 8 μg/ml, 9 μg/ml, 10 μg/ml, 50 μg/ml, 100 μg/ml, 150 μg/ml, 200 μg/ml, 250 μg/ml, 300 μg/ml, 350 μg/ml, 400 μg/ml, 450 μg/ml, 500 μg/ml, 1, or more.

In some embodiments, the mixture of precipitated magnetic particles and the protease can be set still at a desired temperature for a predetermined time. In some embodiments, the desired temperature is the preferred enzymatic reaction temperature of the protease. In some embodiments, the protease is protease K, and the temperature is about 20° C. to about 60° C. In some embodiments, the temperature is about 50° C. to about 60° C. In some embodiments, the temperature is about 55° C. (±2° C.).

In some embodiments, the mixture of precipitated magnetic particles and the protease can be set still for a predetermined period of time. In some embodiments, the time is about 5 min, about 10 min, about 15 min, about 20 min, about 25 min, about 30 min, about 35 min, about 40 min, about 45 min, about 50 min, about 55 min, about 60 min, about 1.5 hour, about 2 hours, about 3 hours, about 4 hours, about 5 hours, or more.

In some embodiments, after the urine sample is pretreated with the magnetic particles and the protease, it is brought to the next stage for DNA extraction. In some embodiments, a lysis solution, a first washing buffer, a second washing buffer, and an elution buffer are used sequentially.

In some embodiments, the lysis solution comprises a compound having the structure of formula (I):

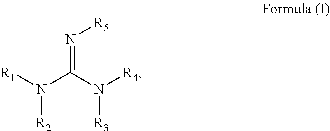

wherein R1, R2, R3, R4, and R5 are independently hydrogen, halogen, acyl, substituted acyl, alkoxycarbonyl, substituted alkoxycarbonyl, aryloxycarbonyl, substituted aryloxycarbonyl, alkyl, substituted alkyl, aryl, substituted aryl, arylalkyl, substituted arylalkyl, heteroaryl, substituted heteroaryl, heteroarylalkyl, substituted heteroarylalkyl, heteroalkyl.

In some embodiments, the compound comprises guanidinium. In some embodiments, the compound comprises guanidinium isothiocyanate, or functional derivatives thereof.

In some embodiments, the lysis solution further comprises a surfactant, a pH buffer, a chelating agent, and an alcohol (e.g., an organic compound in which the hydroxyl functional group (—OH) is bound to acarbon). In some embodiments, the surfactant is Triton X 100. In some embodiments, the pH buffer is Tris-HCl. In some embodiments, the chelating agent is EDTA. In some embodiments, the alcohol is isopropanol.

In some embodiments, the lysis solution has a pH of about 6.2 to 6.8, such as about 6.2, about 6.3, about 6.4, about 6.5, about 6.6, about 6.7, or about 6.8.

In some embodiments, a lysis solution of the present disclosure can be in a concentrated status before it is added to a sample containing DNA (e.g. a liquid sample), such as 2×, 3×, 4×, 5×, 6×, 7×, 8×, 9×, 10×, 15×, 20×, 25×, 30×, 40×, 50×, 60×, 70×, 80×, 90×, 100×, or more, depending on the dilution scales. In some embodiments, the dilution scale can be 10:1, 9:1, 8:1, 7:1, 6:1, 5:1, 4:1, 3:1, 2:1, 1:1, 1:2, 1:3, 1:4, 1:5, 1:6, 1:7, 1:8, 1:9, 1:10, 1:15, 1:20, 1:25, 1:30, 1:40, 1:50, 1:60, 1:70, 1:80, 1:90, 1:99, and so on. Based on the dilution scale, the lysis solution is mixed with a sample containing DNA, so that the final working concentration of 1× is achieved.

In some embodiments, the dilution scale is 3:1 (e.g., 3 volumes of the lysis solution is added to 1 volume of a sample containing DNA). In this case, the preparation of lysis solution comprises a) preparing a solution comprising about 2-6 M guanidinium isothiocyanate, about 1% to about 5% Triton X 100, about 20 mM to about 50 mM Tris-HCl, about 10 to about 50 mM EDTA; and b) adding to the solution about 50% to about 200% (v/v) dosage of isopropanol.

In some embodiments, after the lysis solution is mixed with a sample containing DNA, the working concentrations (1×) of each component are:
(a) about 1.0 M to 5.0M guanidinium isothiocyanate, such as about 1.0 M, about 1.5 M, about 2.0 M, about 2.5 M, about 3.0 M, about 3.5 M, about 4.0 M, about 4.5M, about 5.0M, or more;
(b) about 0.5% to about 4% Triton X-100, such as about 0.5%, about 0.75%, about 1.0%, about 1.25%, about 1.5%, about 1.75%, about 2.0%, about 2.25%, about 2.55, about 2.75%, about 3.0%, about 3.255, about 3.5%, about 3.75%, about 4%, or more;
(c) about 5 mM to about 30 mM Tris-HCl, such as about 5 mM, about 10 mM, about 15 mM, about 20 mM, about 25 mM, about 30 mM, or more;
(d) about 2 mM to about 20 mM EDTA, such as about 2 mM, about 5 mM, about 8 mM, about 11 mM, about 14 mM, about 17 mM, about 20 mM, or more;
(e) about 30% to about 150% (v/v) isopropanol, such as about 30%, about 35%, about 40%, about 45%, about 50%, about 55%, about 60%, about 65%, about 70%, about 75%, about 80%, about 85%, about 90%, about 95%, about 100%, about 105%, about 110%, about 115%, about 120%, about 125%, about 130%, about 135%, about 140%, about 145%, about 150%, or more.

In some embodiments, after the sample containing the magnetic particles is mixed with the lysis solution, a container holding the mixture is shaken for a predetermined time. In some embodiments, the container is shaken for about 10 to 20 min, such as about 10 min, about 11 min, about 12 min, about 13 min, about 14 min, about 15 min, about 16 min, about 17 min, about 18 min, about 19 min, about 20 min, or more.

In some embodiments, after the sample containing the magnetic particles is lysed by the lysis solution of the present disclosure, magnetic particles in the sample are collected by using a magnetic object, such as a magnetic frame or an automatic nucleic acid extraction instrument.

In some embodiments, the collected magnetic particles are washed in a first washing buffer (washing buffer I).

In some embodiments, the first washing buffer comprises a compound having the structure of formula (I)

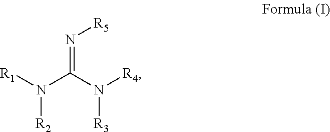

wherein R1, R2, R3, R4, and R5 are independently hydrogen, halogen, acyl, substituted acyl, alkoxycarbonyl, substituted alkoxycarbonyl, aryloxycarbonyl, substituted aryloxycarbonyl, alkyl, substituted alkyl, aryl, substituted aryl, arylalkyl, substituted arylalkyl, heteroaryl, substituted heteroaryl, heteroarylalkyl, substituted heteroarylalkyl, heteroalkyl. In some embodiments, the compound comprises guanidinium. In some embodiments, the compound comprises guanidinium isothiocyanate, or functional derivatives thereof.

In some embodiments, the first washing buffer further comprises a pH buffer, a salt, and an alcohol (e.g., an organic compound in which the hydroxyl functional group (—OH) is bound to a carbon).

In some embodiments, the pH buffer is Tris-HCl. In some embodiments, the salt is a sodium salt, such as NaCl. In some embodiments, the alcohol is ethanol.

In some embodiments, the first washing buffer has a pH of about 4.5 to 5.5, such as about 4.5, about 4.6, about 4.7, about 4.8, about 4.9, about 5.0, about 5.0, about 5.1, about 5.2, about 5.3, about 5.4, about 5.5.

In some embodiments, the first washing buffer of the present disclosure can be in a concentrated status before it is used to wash the magnetic particles, such as 2×, 3×, 4×, 5×, 6×, 7×, 8×, 9×, 10×, 15×, 20×, 25×, 30×, 40×, 50×, 60×, 70×, 80×, 90×, 100×, or more, depending on the dilution scales. In some embodiments, the dilution scale can be 10:1, 9:1, 8:1, 7:1, 6:1, 5:1, 4:1, 3:1, 2:1, 1:1, 1:2, 1:3, 1:4, 1:5, 1:6, 1:7, 1:8, 1:9, 1:10, 1:15, 1:20, 1:25, 1:30, 1:40, 1:50, 1:60, 1:70, 1:80, 1:90, 1:99, and so on. Based on the dilution scale, the washing buffer is diluted by a suitable solvent, so that the final working concentration is achieved.

The working concentrations of each component are:
(a) about 50 to about 100 mM guanidinium isothiocyanate, such as about 50 mM, about 55 mM, about 60 mM, about 65 mM, about 70 mM, about 75 mM, about 80 mM, about 85 mM, about 90 mM, about 95 mM, about 100 mM, or more;
(b) about 20 mM to about 50 mM Tris-HCl, such as about 20 mM, about 25 mM, about 30 mM, about 35 mM, about 40 mM, about 45 mM, about 50 mM, or more;
(c) about 50 mM to about 200 mM NaCl, such as about 50 mM, about 55 mM, about 60 mM, about 65 mM, about 70 mM, about 75 mM, about 80 mM, about 85 mM, about 90 mM, about 95 mM, about 100 mM, about 105 mM, about 110 mM, about 115 mM, about 120 mM, about 125 mM, about 130 mM, about 135 mM, about 140 mM, about 145 mM, about 150 mM, about 155 mM, about 160 mM, about 165 mM, about 170 mM, about 175 mM, about 180 mM, about 185 mM, about 190 mM, about 195 mM, about 200 mM, or more; and
(d) about 40% to about 60% (v/v) ethanol, such as about 40%, about 45%, about 50%, about 55%, about 60%, or more.

In some embodiments, for each 0.1 mg to 1 mg magnetic particles, about 500 to 1000 μl first washing buffer is used.

In some embodiments, the magnetic particles in the sample are washed for a predetermined period of time. In some embodiments, the magnetic particles are washed for about 1 to 10 min, such as about 1 min, about 2 min, about 3 min, about 4 min, about 5 min, about 6 min, about 7 min, about 8 min, about 9 min, about 10 min, or more.

After the magnetic particles have been washed in the first washing buffer, the magnetic particles are collected again by using a magnetic object, such as a magnetic frame or an automatic nucleic acid extraction instrument.

In some embodiments, the collected magnetic particles are washed in a second washing buffer (washing buffer II).

In some embodiments, the second washing buffer further comprises a pH buffer, and an alcohol (e.g., an organic compound in which the hydroxyl functional group (—OH) is bound to a carbon).

In some embodiments, the pH buffer is Tris-HCl. In some embodiments, the alcohol is ethanol.

In some embodiments, the second washing buffer has a pH of about 5.5 to 6.5, such as about 5.5, about 5.6, about 5.7, about 5.8, about 5.9, about 6.0, about 6.1, about 6.2, about 6.3, about 6.4, about 6.5.

In some embodiments, the second washing buffer of the present disclosure can be in a concentrated status before it is used to wash the magnetic particles, such as 2×, 3×, 4×, 5×, 6×, 7×, 8×, 9×, 10×, 15×, 20×, 25×, 30×, 40×, 50×, 60×, 70×, 80×, 90×, 100×, or more, depending on the dilution scales. In some embodiments, the dilution scale can be 10:1, 9:1, 8:1, 7:1, 6:1, 5:1, 4:1, 3:1, 2:1, 1:1, 1:2, 1:3, 1:4, 1:5, 1:6, 1:7, 1:8, 1:9, 1:10, 1:15, 1:20, 1:25, 1:30, 1:40, 1:50, 1:60, 1:70, 1:80, 1:90, 1:99, and so on. Based on the dilution scale, the washing buffer is diluted by a suitable solvent, so that the final working concentration is achieved.

The working concentrations of each component are:
(a) about 10 mM to about 50 mM Tris-HCl, such as about 10 mM, about 15 mM, about 20 mM, about 25 mM, about 30 mM, about 35 mM, about 40 mM, about 45 mM, about 50 mM, or more; and
(b) about 70% to 80% ethanol, such as about 71%, about 72%, about 72%, about 73%, about 74%, about 75%, about 76%, about 77%, about 78%, about 79%, about 80%.

In some embodiments, for each 0.1 mg to 1 mg magnetic particles, about 500 to 1000 μl second washing buffer is used.

In some embodiments, the magnetic particles in the sample are washed in the second washing buffer for a predetermined period of time. In some embodiments, the magnetic particles are washed for about 1 to 10 min, such as about 1 min, about 2 min, about 3 min, about 4 min, about 5 min, about 6 min, about 7 min, about 8 min, about 9 min, about 10 min, or more.

In some embodiments, after being washed with the second washing buffer, the magnetic particles are collected again by using a magnetic object, such as a magnetic frame or an automatic nucleic acid extraction instrument.

In some embodiments, the collected magnetic particles are treated in an elution buffer to release the isolated DNA molecules.

In some embodiments, the elution buffer is a TE buffer. In some embodiments, the TE buffer is a 1×TE buffer comprises about 10 mM Tris and about 1 mM EDTA. In some embodiments, the pH of the TE buffer is brought to about 8.0 with HCl.

In some embodiments, before the magnetic particles are treated by the elution buffer, they are set still for a predetermined time at a preselected temperature.

In some embodiments, the predetermined time is about 1 to 10 min, such as about 1 min, about 2 min, about 3 min, about 4 min, about 5 min, about 6 min, about 7 min, about 8 min, about 9 min, about 10 min, or more.

In some embodiments, the preselected temperature can be room temperature, a higher or a lower temperature, such as about −80° C. to about 37° C.

In some embodiments, the washing-off step comprises heating the elution buffer containing the magnetic particles at a relevantly high temperature, such as about 50° C. to about 75° C., such as about 50° C., about 55° C., about 60° C., about 65° C., about 70° C., about 75° C., or more.

### Compositions, Chips, and Kits

The present disclosure further provides nucleotide arrays for detecting and/or identifying HPV genotypes. In some embodiments, the nucleotide arrays are DNA array, RNA array, or mixture thereof. In some embodiments, the nucleotide arrays are DNA microarray. As used herein, a DNA microarray (also commonly known as DNA chip or biochip) is a collection of microscopic DNA spots attached to a solid surface. The nucleotide arrays can comprise DNA molecules comprising one or more primers/probe of the present disclosure.

In some embodiments, arrays of the present disclosure are arrangements of addressable locations on a substrate, with each address containing a nucleic acid, such as a probe. In some embodiments, each address corresponds to a single type or class of nucleic acid, such as a single probe, though a particular nucleic acid may be redundantly contained at multiple addresses. The arrays can be either microarrays or macroarrays. A microarray is a miniaturized array requiring microscopic examination for detection of hybridization. Larger macroarrays allow each address to be recognizable by the naked human eye and, in some embodiments, a hybridization signal is detectable without additional magnification. The addresses may be labeled, keyed to a separate guide, or otherwise identified by location.

The use of the term “array” includes the arrays found in DNA microchip technology. As one, non-limiting example, the probes could be contained on a DNA microchip similar to the GENECHIP® products and related products commercially available from Affymetrix, Inc. (Santa Clara, Calif.).

The present disclosure further provides kits. In some embodiments, the kit may be a kit for the amplification, detection, identification or quantification of HPV sequences in a sample. The kit may comprise a pair of primers (e.g., a forward primer, a reverse primer), and a corresponding probe as described herein.

A kit of the present disclosure can comprise polynucleotides of the present disclosure as described herein. In some embodiments, the kit may also comprise reagents and/or devices for extracting DNA from a sample, such as a urine sample, as described herein. The kit can also be used for predicting cancer in a subject in need thereof.

In some embodiments, the kits may comprise a polynucleotide described herein together with any or all of the following: assay reagents, buffers, probes and/or primers, and assay solution, or another pharmaceutically acceptable emulsion and suspension base. In addition, the kits may include instructional materials containing directions (e.g., protocols) for the practice of the methods described herein. The kits may further comprise a software package for data analysis.

Any of the compositions described herein may be comprised in a kit. In a non-limiting example, reagents for isolating, labeling, and/or evaluating a DNA and/or RNA populations are included in a kit. It may also include one or more buffers, such as reaction buffer, labeling buffer, washing buffer, or a hybridization buffer, compounds for preparing the DNA sample, components hybridization and components for isolating DNA.

In some embodiments, the kits comprise containers such as solution container or reaction tubes. The containers in which the nucleic acids are supplied can be any conventional container that is capable of holding the supplied form, for instance, microfuge tubes, ampoules, or bottles. The kits can include either labeled or unlabeled nucleic acid probes for use in detection, typing, and subtyping of HPV.

In some applications, one or more primers and/or probes as described herein, such as pairs of primers and/or their corresponding probe may be provided in pre-measured single use amounts in individual, typically disposable, tubes or equivalent containers. With such an arrangement, the sample to be tested for the presence of HPV can be added to the individual tubes and amplification carried out directly.

The amount of primers and probes supplied in the kit can be any appropriate amount, and may depend on the target market to which the product is directed. For instance, if the kit is adapted for research or clinical use, the amount of each nucleic acid primer provided would likely be an amount sufficient to prime several PCR amplification reactions.

In some embodiments, kits also may include the reagents necessary to carry out PCR amplification reactions, including DNA sample preparation reagents, appropriate buffers (such as polymerase buffer), salts (for example, magnesium chloride), and deoxyribonucleotides (dNTPs).

One or more reference control sequences for use in the PCR reactions also may be supplied in the kit (for example, for the detection of a control gene, such as β-actin).

The kits may also comprise a positive and/or a negative control sample (e.g., a sample that comprise or does no comprise DNA of one or more given HPV subtypes).

### Definition

References to “one embodiment”, “an embodiment”, “one example”, and “an example” indicate that the embodiment(s) or example(s) so described may include a particular feature, structure, characteristic, property, element, or limitation, but that not every embodiment or example necessarily includes that particular feature, structure, characteristic, property, element or limitation. Furthermore, repeated use of the phrase “in one embodiment” does not necessarily refer to the same embodiment, though it may.

As used herein, the term “biological sample” means cells, tissues, an organ biopsy, a tissue biopsy, a body fluid, a body secretion, a culture or culture medium, an aqueous solution, an emulsion, a dispersion, a suspension that contains isolated and purified pathogen, or components thereof; unfixed, frozen, fixed sample in formalin and/or embedded in paraffin. A biological sample may have already been subjected to purification steps, but it may also be present in an unpurified form. In some embodiments, the biological sample is amniotic fluid, aqueous humour and vitreous humour, bile, blood, blood plasma, blood serum, cerebrospinal fluid, cerumen (earwax), chyle, chime, endolymph and perilymph, exudates, feces, female ejaculate, gastric acid, gastric juice, lymph, mucus (including nasal drainage and phlegm), pericardial fluid, peritoneal fluid, pleural fluid, pus, rectal discharge, rheum, saliva, sebum (skin oil), serous fluid, semen, serum, smegma, sputum, synovial fluid, sweat, tears, urine, milk, skin scrapes, prostate fluid, surface washings, vaginal secretion, bone marrow aspirates, bronchoalveolar lavage, tracheal aspirates, nasopharyngeal aspirates, vaginal discharge, vomit, oropharyngeal aspirates, or any mixture thereof.

“Nucleic acid” or “oligonucleotide” or “polynucleotide”, as used herein means at least two nucleotides covalently linked together. The depiction of a single strand also defines the sequence of the complementary strand. Thus, a nucleic acid also encompasses the complementary strand of a depicted single strand. Many variants of a nucleic acid may be used for the same purpose as a given nucleic acid. Thus, a nucleic acid also encompasses substantially identical nucleic acids and complements thereof. A single strand provides a probe that may hybridize to a target sequence under stringent hybridization conditions. Thus, a nucleic acid also encompasses a probe that hybridizes under stringent hybridization conditions. Nucleic acids may be single stranded or double stranded, or may contain portions of both double stranded and single stranded sequences. The nucleic acid may be DNA, both genomic and cDNA, RNA, or a hybrid, where the nucleic acid may contain combinations of deoxyribo- and ribo-nucleotides, and combinations of bases including uracil, adenine, thymine, cytosine, guanine, inosine, xanthine hypoxanthine, isocytosine and isoguanine Nucleic acids may be obtained by chemical synthesis methods or by recombinant methods.

“Stringent hybridization conditions” as used herein mean conditions under which a first nucleic acid sequence (e.g., probe) will hybridize to a second nucleic acid sequence (e.g., target), such as in a complex mixture of nucleic acids. Stringent conditions are sequence-dependent and will be different in different circumstances. Stringent conditions may be selected to be about 5-10° C. lower than the thermal melting point (Tₘ) for the specific sequence at a defined ionic strength pH. The Tₘ may be the temperature (under defined ionic strength, pH, and nucleic concentration) at which 50% of the probes complementary to the target hybridize to the target sequence at equilibrium (as the target sequences are present in excess, at Tₘ, 50% of the probes are occupied at equilibrium). Stringent conditions may be those in which the salt concentration is less than about 1.0 M sodium ion, such as about 0.01-1.0 M sodium ion concentration (or other salts) at pH 7.0 to 8.3 and the temperature is at least about 30° C. for short probes (e.g., about 10-50 nucleotides) and at least about 60° C. for long probes (e.g., greater than about 50 nucleotides). Stringent conditions may also be achieved with the addition of destabilizing agents such as formamide. For selective or specific hybridization, a positive signal may be at least 2 to 10 times background hybridization. Exemplary stringent hybridization conditions include the following: 50% formamide, 5×SSC, and 1% SDS, incubating at 42° C., or, 5×SSC, 1% SDS, incubating at 65° C., with wash in 0.2×SSC, and 0.1% SDS at 65° C.

“Variant” as used herein referring to a nucleic acid means (i) a portion of a referenced nucleotide sequence; (ii) the complement of a referenced nucleotide sequence or portion thereof; (iii) a nucleic acid that is substantially identical to a referenced nucleic acid or the complement thereof; or (iv) a nucleic acid that hybridizes under stringent conditions to the referenced nucleic acid, complement thereof, or a sequence substantially identical thereto.

“Substantially complementary” as used herein means that a first sequence is at least 60%, 65%, 70%, 75%, 80%, 85%, 90%, 95%, 97%, 98% or 99% identical to the complement of a second sequence over a region of 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100 or more nucleotides, or that the two sequences hybridize under stringent hybridization conditions.

“Substantially identical” as used herein means that a first and a second sequence are at least 60%, 65%, 70%, 75%, 80%, 85%, 90%, 95%, 97%, 98% or 99% identical over a region of 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100 or more nucleotides or amino acids, or with respect to nucleic acids, if the first sequence is substantially complementary to the complement of the second sequence.

As used herein the term “diagnosing” refers to classifying pathology, or a symptom, determining a severity of the pathology (e.g., grade or stage), monitoring pathology progression, forecasting an outcome of pathology and/or prospects of recovery.

As used herein the phrase “subject in need thereof” refers to an animal or human subject who is known to have cancer, at risk of having cancer (e.g., a genetically predisposed subject, a subject with medical and/or family history of cancer, a subject who has been exposed to carcinogens, occupational hazard, environmental hazard) and/or a subject who exhibits suspicious clinical signs of cancer (e.g., blood in the stool or melena, unexplained pain, sweating, unexplained fever, unexplained loss of weight up to anorexia, changes in bowel habits (constipation and/or diarrhea), tenesmus (sense of incomplete defecation, for rectal cancer specifically), anemia and/or general weakness). Additionally or alternatively, the subject in need thereof can be a healthy human subject undergoing a routine well-being check up.

As used herein the term “about” refers to ±10%.

The phrase “consisting essentially of” means that the composition or method may include additional ingredients and/or steps, but only if the additional ingredients and/or steps do not materially alter the basic and novel characteristics of the claimed composition or method.

As used herein, the singular form “a”, “an” and “the” include plural references unless the context clearly dictates otherwise. For example, the term “a compound” or “at least one compound” may include a plurality of compounds, including mixtures thereof.

The word “exemplary” is used herein to mean “serving as an example, instance or illustration”. Any embodiment described as “exemplary” is not necessarily to be construed as preferred or advantageous over other embodiments and/or to exclude the incorporation of features from other embodiments.

The word “optionally” is used herein to mean “is provided in some embodiments and not provided in other embodiments”. Any particular embodiment of the invention may include a plurality of “optional” features unless such features conflict.

As used herein, “dosage of isopropanol (v/v)” means the ratio of the volume of isopropanol to the volume of the solution comprising all other components in the solution during the preparation of the final solution. For example, “isopropanol has a dosage of about 50% to 200% (v/v)” means that, when preparing the final solution, the volume of added isopropanol is about 50% to 200% of the volume of the solution comprising all other components in the final solution.

Certain embodiments of the present disclosure are further described in the following Examples. It should be understood that these Examples are given by way of illustration only. From the above discussion and these Examples, one skilled in the art can ascertain the essential characteristics, and without departing from the spirit and scope thereof, can make various changes and modifications of the embodiments of the invention to adapt it to various usages and conditions. Thus, various modifications of the embodiments of the invention, in addition to those shown and described herein, will be apparent to those skilled in the art from the foregoing description. Such modifications are also intended to fall within the scope of the appended claims.

### Examples

### Example 1: Primers/Probes Design and Test

Snijders et al. (J. Gen. Virol., 71 (1990), 173 to 181) and Surentheran et al., (J. Clin. Path., 51 (1998), 606-610) describe a PCR process for detecting HPV L1 gene DNA. A disadvantage of the two methods is that only a limited number of HPV types can be detected. For example, the primers described by Snijder et al. can only detect some of the HPV types, such as HPV30, HPV39, and HPV51, but with greatly reduced sensitivity. In addition, when the primers described by Snijder et al. are used, some HPV types, such as HPV18, result in the formation of additional bands. Thus, the existing tests only detect a limited spectrum of HPV subtypes, and some rare HPV subtypes cannot be adequately detected. The present disclosure provides compositions and methods that can be used to detect and/or genotype as many as 14 high-risk HPV subtypes and two low-risk HPV subtypes in a single test tube or two test tubes.

Design Primers and Probes

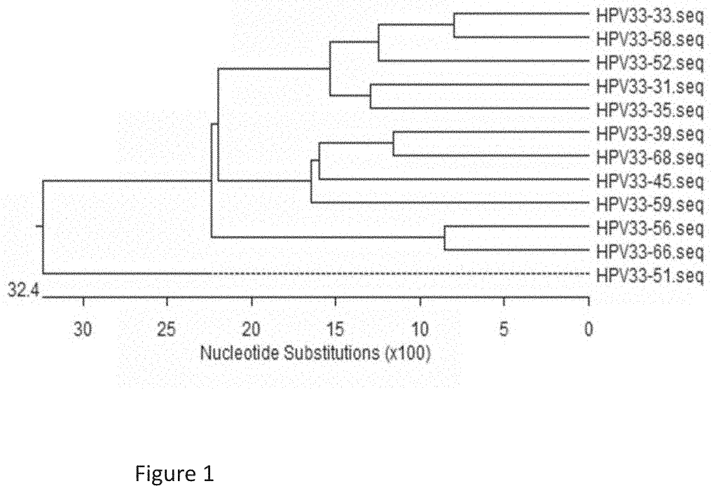

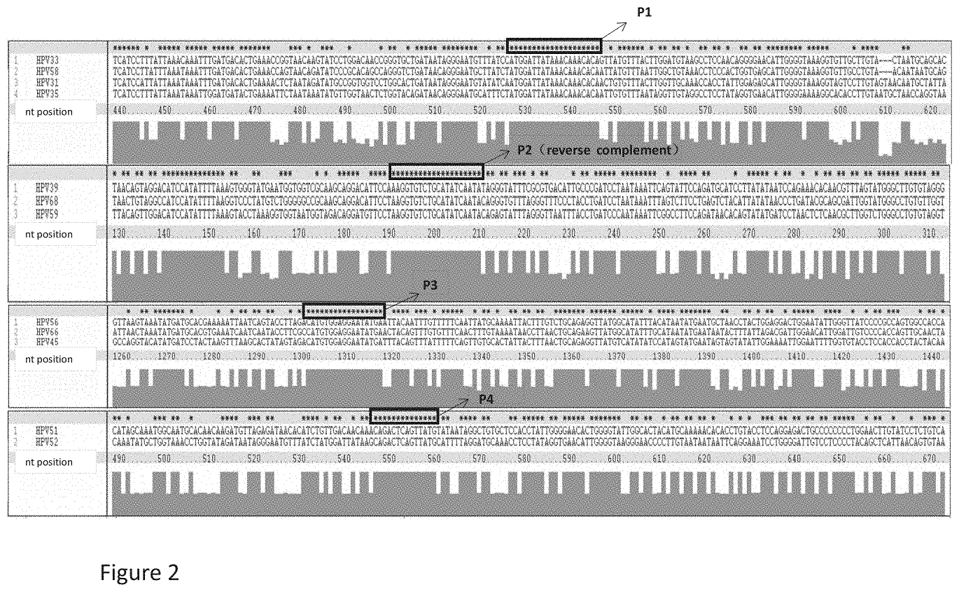

L1 gene sequences in several HPV subtypes (HPV16, HPV18, HPV31, HPV33, HPV35, HPV39, HPV45, HPV51, HPV52, HPV56, HPV58, HPV59, HPV66, HPV68a, HPV68b, HPV6, HPV11 and HPV17), and other common HPV types (HPV26, HPV40, HPV42, HPV43, HPV44, HPV53, HPV54, HPV61, HPV67, HPV69, HPV70, HPV71, HPV72, HPV73, HPV81, HPV82, HPV83) are obtained from National Center for Biotechnology Information (NCBI). The DNAstart® software was used to compare L1 gene sequences in 12 high-risk HPVs (HPV31, HPV33, HPV35, HPV39, HPV45, HPV51, HPV52, HPV56, HPV58, HPV59, HPV66, and HPV68). According to the phylogenetic tree (FIG. 1), the 12 high-risk HPVs were divided into 4 categories: Class 1 (HPV31, HPV33, HPV35, 58), Class 2 (HPV39, HPV59, HPV68), Class 3 (HPV45, HPV56, HPV66), Class 4 (HPV51, HPV52). Then HPV L1 genes of these four classes were aligned using DNAstart® software in order to discover conserved regions. Based on the conserved regions, probes P1, P2, P3, and P4 (SEQ ID NOs: 37, 38, 39, and 40) were designed for detection of these 12 HPV subtypes (FIG. 2).

A software was used to design primers and probes for HPV16, HPV18, HPV6, HPV11 and these 12 high-risk HPVs. For each design, there were several possible sets of specific primers or probes. The software was used to determine dimers that may be formed between the primers and probes in each design, and primer/probe combinations that had the least possible dimer formation rate were selected.

Primers and Probes Test

According to the optimal primer probe sequence obtained by software design analysis, the corresponding primers and probes were synthesized and tested in a real-time fluorescent quantitative PCR reaction solution. The reaction solution was used to amplify template DNA. The template DNA used included 33 synthetic HPV L1 genes (cloned into pUC57 vector). The real-time PCR system used included: 1×buffer (invitrogen), 0.2 mM dNTP (invitrogen), 3 mM MgCL2 (invitrogen), 0.2 μM-0.6 μM primer and probe, and 1 U Taq enzyme (invitrogen Platinum™ Taq). The instrument used for the fluorescent quantitative PCR was Roche Lightcycler 480II. The qPCR reaction conditions were as follows:

| Temperature | Time | | Cycles |
|---|---|---|---|
| 94° C. | 2 | min | 1 |
| 94° C. | 20 | s | 5 |
| 63° C. | 1 | min | (touch down, |
| | | | reduce 1° C. per cycle) |
| 94° C. | 20 | s | 40 |
| 58° C. | 1 | min | |

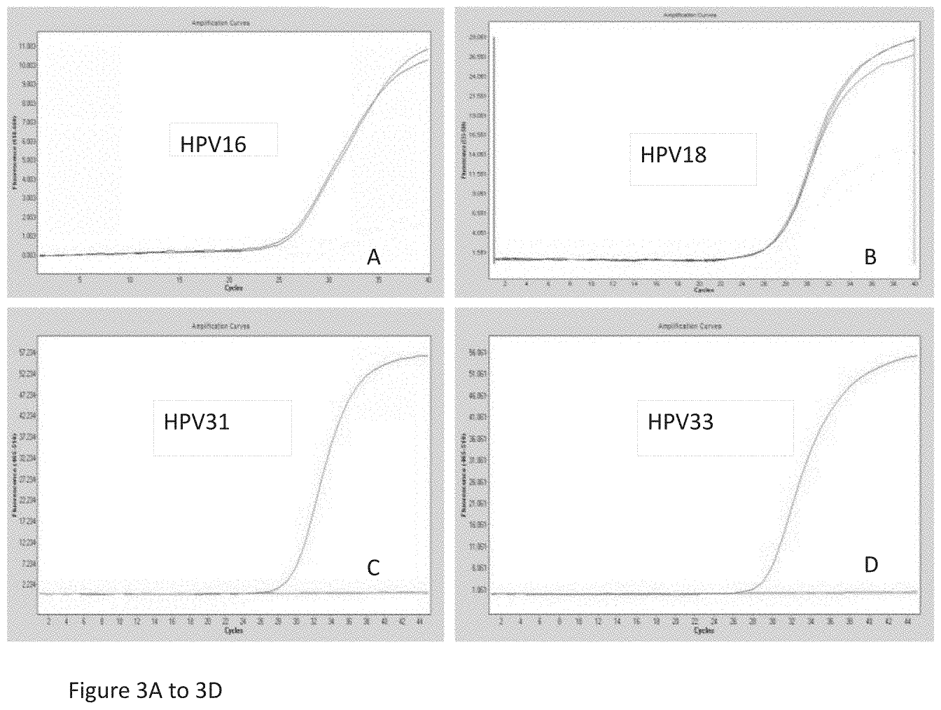

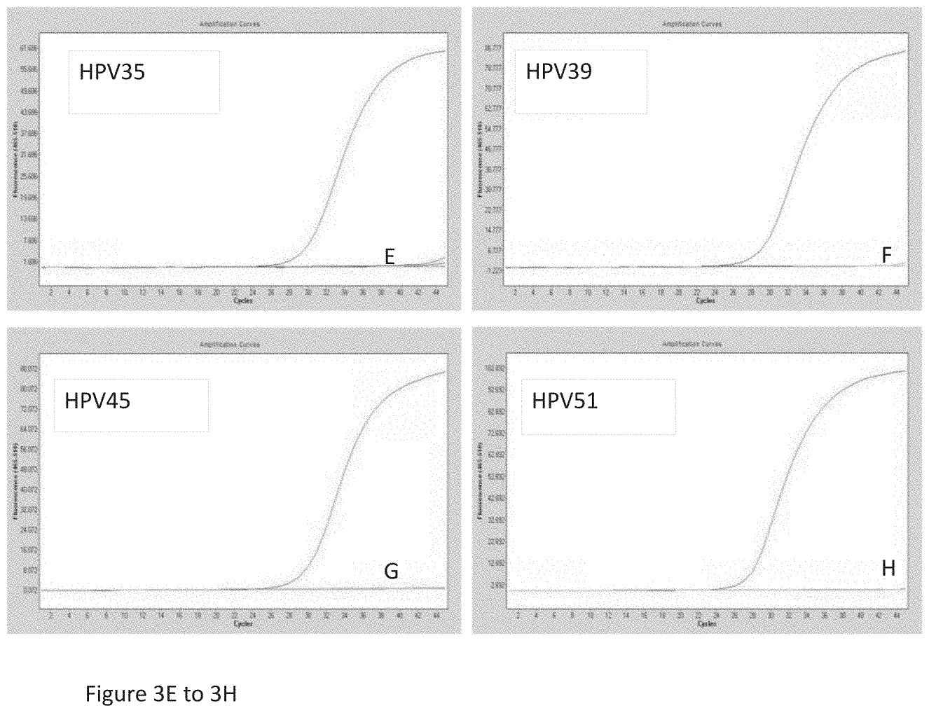

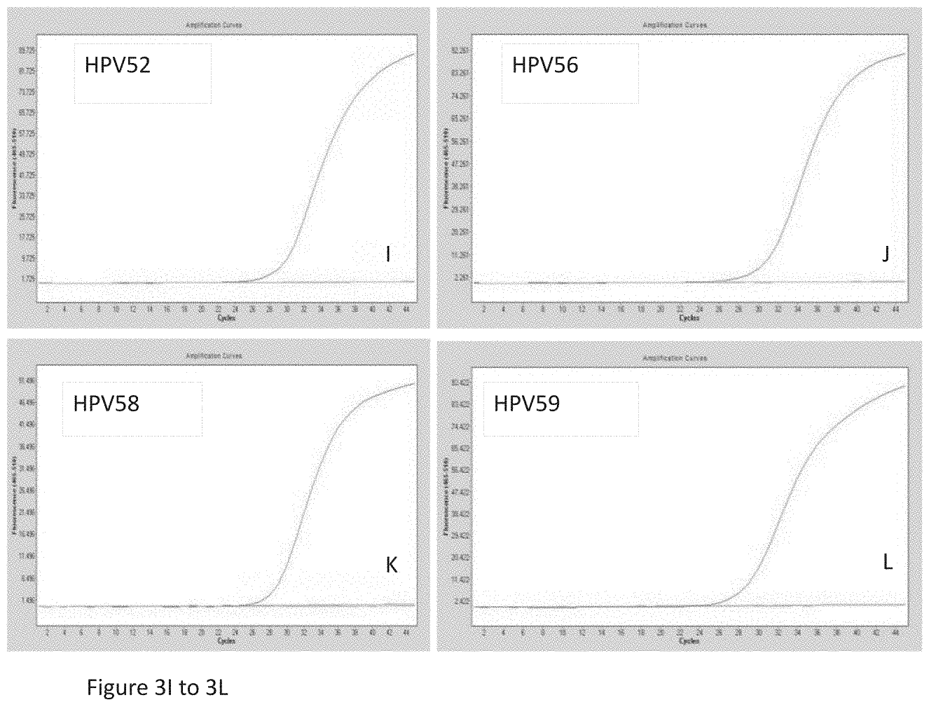

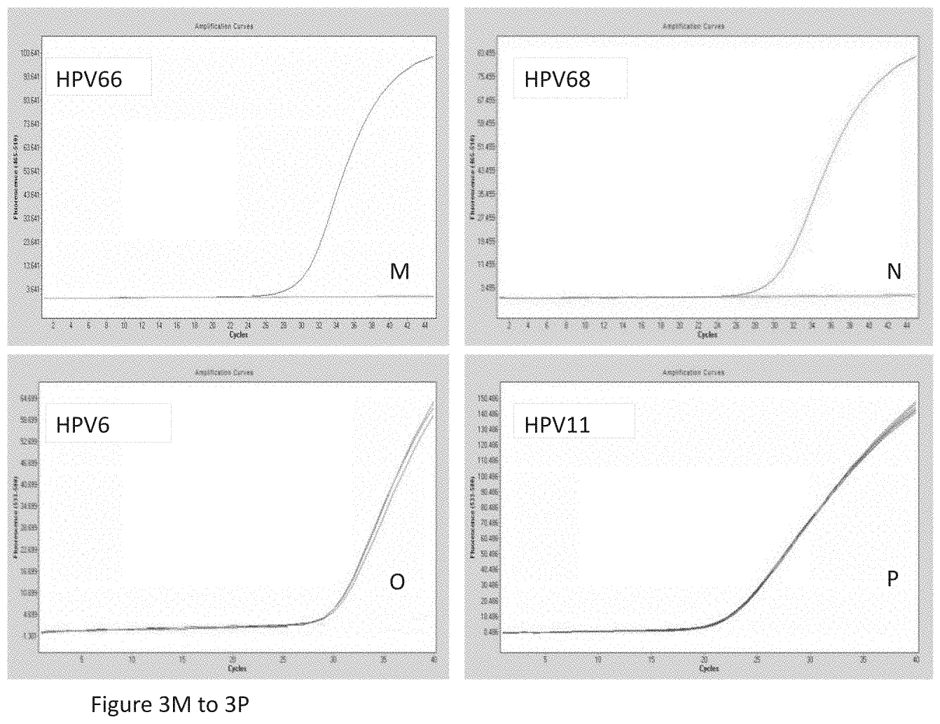

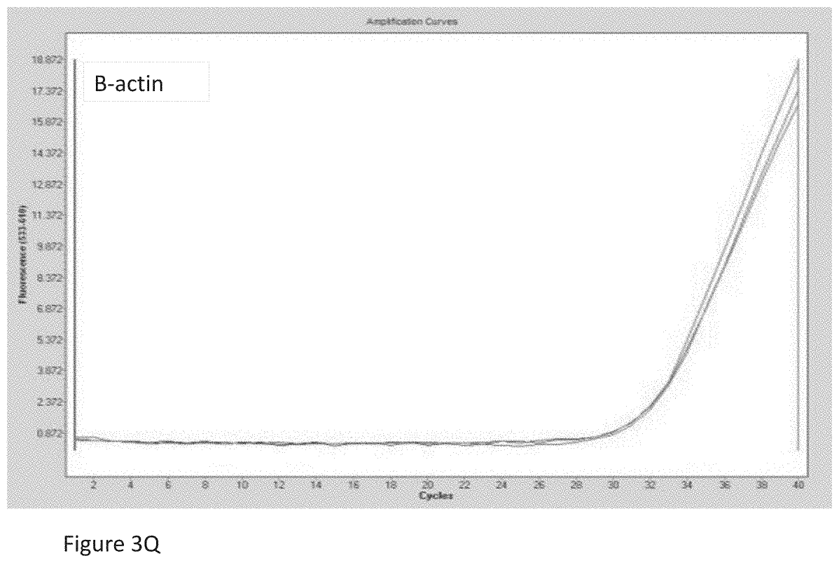

Among all primers and probes tested, the combinations of primers and probes that provided the best results were selected, and their amplification curves for each HPV subtype L1 gene are shown in FIGS. 3A to 3P. FIG. 3Q shows the amplification curve of β-actin control gene using the most optimized primer/probe set.

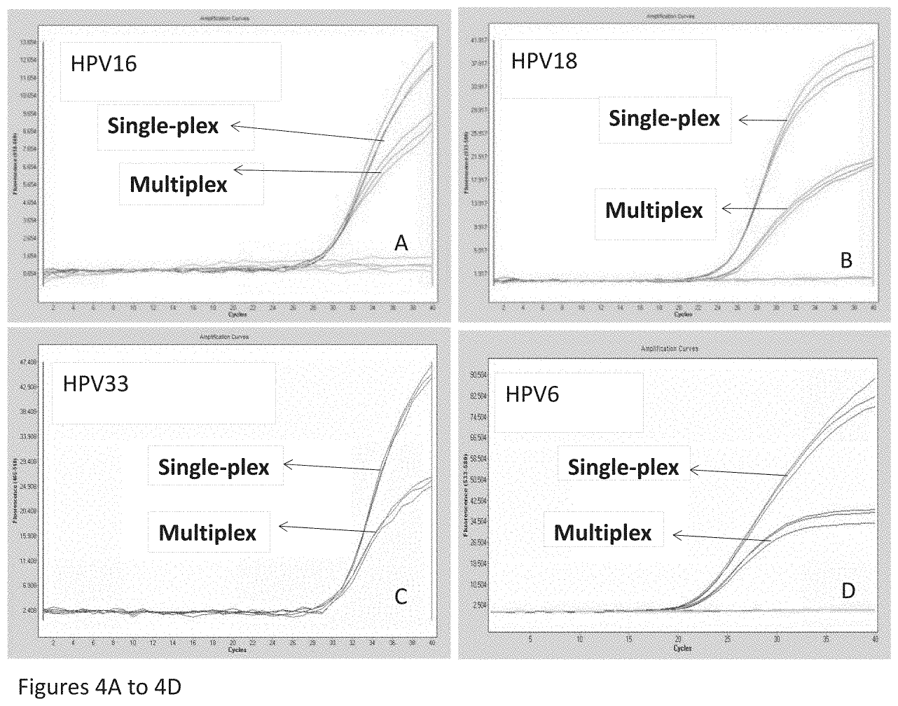

Next, we tested when these preferred primers/probes are used in a multiplex PCR system to amplify all HPV subtype sequences, whether the amplification results of any certain HPV subtype(s) will be worse than a single-plex PCR system in which only a single set of primer/probe is used to amplify one specific HPV subtype sequence. To do that, HPV L1 template DNA for each HPV subtype was used in either a series of single-plex PCRs (each containing two primers and one probe specific for a single HPV subtype L1 gene) or a multiplex PCR (containing primers and probes for all 16 HPV subtypes, i.e., PHV16, HPV18, HPV31, HPV33, HPV35, HPV39, HPV45, HPV51, HPV52, HPV56, HPV58, HPV59, HPV66, HPV68, HPV6, and HPV11). As shown in FIGS. 4A to 4B, except for HPV18, amplifications of HPV L1 sequences in the multiplex PCR and amplifications in each single-plex PCR did not have significant difference (as demonstrated by HPV16, HPV33, and HPV6).

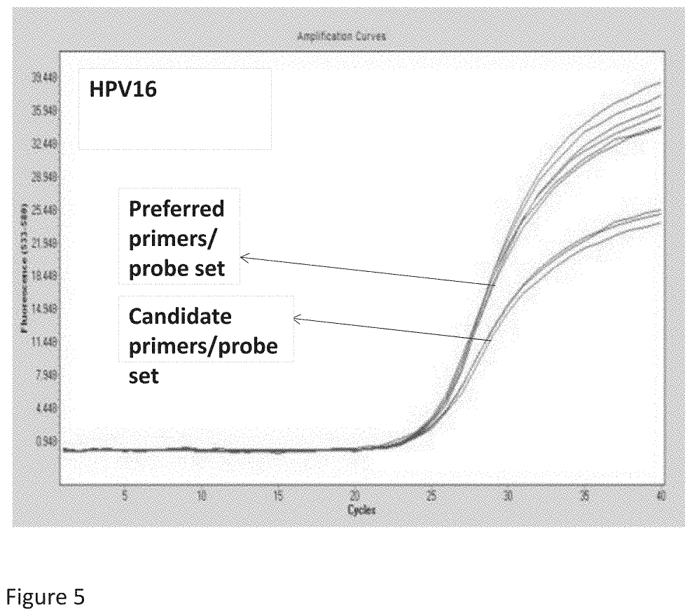

Primers and probes disclosed herein for each HPV subtype are superior over other primers and probes. For example, the preferred primers and probe for HPV16 (SEQ ID No: 1, SEQ ID NO: 2, and SEQ ID NO: 37) were compared to a candidate set of primers and probe provided by software (candidate forward primer SEQ ID NO: 46, TCCAGATTATATTAAAATGGTGTCAGAACC; candidate reverse primer SEQ ID NO: 47, GACCCAGAGCCTTTAATGTATAAATCG, and candidate probe SEQ ID NO: 48, 5′-CY5-ACATTTTCACCAACAGCACCAGCCCTATT3′-BHQ2). Both the preferred set and the candidate set were used to prepare a multiplex qPCR system for detection of all 14 high-risk HPVs (PHV16, HPV18, HPV31, HPV33, HPV35, HPV39, HPV45, HPV51, HPV52, HPV56, HPV58, HPV59, HPV66, HPV68). The same amount of HPV16 template DNA was used in each multiplex qPCR system. The results as shown in FIG. 5 indicate that the preferred set of primers and probe for HPV16 provided better HPV16 amplification compared to the candidate set.

### Example 2: High-Risk HPV Detection Kit

As a non-limiting example, a typical kit can comprise the following parts:
(1) High-risk HPV qPCR mixture: 1×buffer (20 mM Tris-HCl, 50 mM KCl, pH8.4), 0.15-0.3 mM dNTP, 2-4 mM MgCl₂, 0.2-1.2 μM primers/probes (14 high-risk types), 0.1 1 mg/ml BSA, 0.2%-2% (V/V) formamide, 0.2 mm˜2 mM spermidine, 10 mM˜30 mM tetramethylammonium chloride, 0.01 mM˜0.1 mM DTT, 0.2%˜2% 2-pyrrolidone and H₂O
(2) Taq enzyme: 1˜6U/μl Taq enzyme
(3) Positive control: high-risk HPV16, HPV18, and HPV45 L1 gene plasmid (each plasmid is 10³ copies/ml), mixed with high-risk HPV DNA-negative urine
(4) Negative control: high-risk HPV DNA negative urine

### Example 3: HPV Detection Kit for 14 High-Risk Subtypes and Two Low-Risk Subtypes

As a non-limiting example, a typical kit for detection of 14 high-risk subtypes and two low-risk subtypes can comprise the following parts:
(1) HPV qPCR mixture I: 1×buffer (20 mM Tris-HCl, 50 mM KCl, pH 8.4), 0.2-0.3 mM dNTP, 2-3 mM MgCl₂, 0.2-0.8 μM primer probe (HPV16, HPV18, HPV35, HPV39, HPV68, HPV59, HPV56, HPV66, HPV51), 0.1˜1 mg/ml BSA, 0.2%-2% (VN) formamide, 0.2 mm˜2 mM spermidine, 10 mM˜30 mM tetramethylammonium chloride, 0.01 mM˜0.1 mM DTT, 0.2%˜2% 2-pyrrolidone and H₂O;
(2) HPV qPCR mixture II: 1×buffer (20 mM Tris-HCl, 50 mM KCl, pH 8.4), 0.2-0.3 mM dNTP, 2-3 mM MgCl₂, 0.2-0.8 μM primer probe (HPV6, HPV11, HPV33, HPV58, HPV31, HPV45, HPV52, β-actin), 0.1˜1 mg/ml BSA, 0.2%-2% (VN) formamide, 0.2 mm˜2 mM spermidine, 10 mM˜30 mM tetramethylammonium chloride, 0.01 mM˜0.1 mM DTT, 0.2%˜2% 2-pyrrolidone and H₂O;
(3) Taq enzyme: 1˜6U/μl Taq enzyme;
(4) Positive control: high-risk HPV16, HPV18, and HPV45 L1 gene plasmid (each plasmid is 10³ copies/ml), and high-risk HPV DNA-negative urine or its DNA;
(5) Negative control: HPV DNA negative urine or its DNA.

### Example 4: High-Risk HPV Detection of Clinical Urine Samples

In total 170 samples came from a community hospital were used for a large scale HPV detection clinical trial.

1. Pretreatment of urine sample: 10 ml of each urine sample was added into a 50 ml centrifuge tube. 20 μl of hydroxyl magnetic beads was added into the sample and mixed by vortexing. The tube was centrifuged for 5 min at 10000 rpm. Afterwards, supernatant was carefully discarded, and 500 μl of pellet was placed in a new 1.5 ml centrifuge tube. 2.5 μl of proteinase K was mixed with the pellet. The tube was heated in a metal bath at 56° C. for 30 min.

2. Extraction reagent dispensing: The lysis solution, washing buffer A, washing buffer B, and the elution buffer were added to a 96-well deep well extraction plate in a volume of 750 μl, 600 μl, 600 μl, and 50 μl, respectively.

Table 3 demonstrated a possible sample loading plan. Among them, for each of the 8 rows A to H, two samples can be held for DNA extraction. For a 96-well plant, DNA from 16 samples can be extracted.

TABLE 3
Sample loading plan for DNA extraction on a 96-well plate.

| | 1 | 2 | 3 | 4 | 5 | 6 | 7 | 8 | 9 | 10 | 11 | 12 |
|---|---|---|---|---|---|---|---|---|---|---|---|---|
| A | Lysis | Lysis | Wash | Wash | | elution | Lysis | Lysis | Wash | Wash | | elution |
| | solution | solution | Buffer | Buffer | | buffer | solution | solution | Buffer | Buffer | | buffer |
| | | | I | II | | | | | I | II | | |
| B | Lysis | Lysis | Wash | Wash | | elution | Lysis | Lysis | Wash | Wash | | elution |
| | solution | solution | Buffer | Buffer | | buffer | solution | solution | Buffer | Buffer | | buffer |
| | | | I | II | | | | | I | II | | |
| C | Lysis | Lysis | Wash | Wash | | elution | Lysis | Lysis | Wash | Wash | | elution |
| | solution | solution | Buffer | Buffer | | buffer | solution | solution | Buffer | Buffer | | buffer |
| | | | I | II | | | | | I | II | | |
| D | Lysis | Lysis | Wash | Wash | | elution | Lysis | Lysis | Wash | Wash | | elution |
| | solution | solution | Buffer | Buffer | | buffer | solution | solution | Buffer | Buffer | | buffer |
| | | | I | II | | | | | I | II | | |
| E | Lysis | Lysis | Wash | Wash | | elution | Lysis | Lysis | Wash | Wash | | elution |
| | solution | solution | Buffer | Buffer | | buffer | solution | solution | Buffer | Buffer | | buffer |
| | | | I | II | | | | | I | II | | |
| F | Lysis | Lysis | Wash | Wash | | elution | Lysis | Lysis | Wash | Wash | | elution |
| | solution | solution | Buffer | Buffer | | buffer | solution | solution | Buffer | Buffer | | buffer |
| | | | I | II | | | | | I | II | | |
| G | Lysis | Lysis | Wash | Wash | | elution | Lysis | Lysis | Wash | Wash | | elution |
| | solution | solution | Buffer | Buffer | | buffer | solution | solution | Buffer | Buffer | | buffer |
| | | | I | II | | | | | I | II | | |
| H | Lysis | Lysis | Wash | Wash | | elution | Lysis | Lysis | Wash | Wash | | elution |
| | solution | solution | Buffer | Buffer | | buffer | solution | solution | Buffer | Buffer | | buffer |
| | | | I | II | | | | | I | II | | |

750 μl of the lysis solution and 250 μl of the above pretreated urine sample were mixed in each well of columns 1, 2, 7, and 8. 600 μl of washing solution A was added into each well of columns 3 and 9. 600 μl of washing buffer B was added into each well of columns 4 and 10. 50 μl of the elution buffer was added into each well of columns 6 and 12.

3. DNA Extraction using an automated DNA extraction equipment: The above described 96-well containing samples were placed into an automated DNA extraction equipment (Xi′An Tian Long, model NP968-S). Based on the manufacture manual, the following program was used:

TABLE 4
Program for automated DNA extraction equipment

| | | Magnetic | | | | | | |
|---|---|---|---|---|---|---|---|---|
| | Mixing | reatment | | Vortexing | | Hole | | |
| Step | time | time | Volume | speed | Tem | position | Description | Waiting time |
| 1 | 10 min | 60 s | 1000 μl | Level 7 | | 1 | Lysis; binding | |
| 2 | 5 min | 60 s | 1000 μl | Level 7 | | 2 | Lysis; binding | |
| 3 | 3 min | 60 s | 600 μl | Level 7 | | 3 | Washing | |
| 4 | 2 min | 60 s | 600 μl | Level 7 | | 4 | Washing | |
| 5 | 5 min | 60 s | 50 μl | Level 7 | 65° C. | 6 | elution | 5 min |
| 6 | 2 min | | 50 μl | Level 6 | | 4 | Remove | |
| | | | | | | | magnetic | |
| | | | | | | | particles | |

4. Preparation and packaging of high-risk HPV qPCR reaction system: To produce a high-risk HPV multiplex qPCR reaction system, 39 μl of “high-risk HPV qPCR reaction solution” and 1 μl of “Taq enzyme” for each sample were mixed, and dispensed in 200 μl PCR tube.

5. Template loading: 10 μl of the extracted DNA template in step 3 above was added to the HPV qPCR reaction system mentioned in the 200 μl PCR tubes using an 8-channel pipette. The PCR tube was centrifuged and ready for PCR.

6. Fluorescence quantitative PCR instrument amplification test: The PCR tube with the template and the reaction solution described above was placed on a fluorescence quantitative PCR instrument for detection. The PCR instrument contains CY5, HEX, FAM, and ROX fluorescence channels, and the PCR program is set as follows:

| Temperature | Time | | Cycles |
|---|---|---|---|
| 94° C. | 2 | min | 1 |
| 94° C. | 20 | s | 5 |
| 63° C. | 1 | min | (touch down, |
| | | | reduce 1° C. per cycle) |
| 94° C. | 20 | s | 40 |
| 58~60° C. | 1 | min | |

The detection results are provided in Table 5 below:

TABLE 5
HPV detection results

| Total | | | Other | | |
|---|---|---|---|---|---|
| samples | | | 12 high- | Mixed | |
| n = 170 | HPV16 | HPV18 | risk subtypes | type | Total |
| Positive | 3 | 1 | 16 | 4 | 24 |
| sample | | | | | |
| number | | | | | |
| Positive rate | 1.76% | 0.59% | 9.41% | 2.40% | 14.11% |

### Example 5: Comparison of High-Risk HPV Tests Using Urine Samples and Cervical Exfoliated Cell Samples

Both urine sample and cervical exfoliated cell sample were collected from 90 human subjects to produce 90 sample sets. Each of the sample set comprise a urine sample and a cervical exfoliated cell sample collected from the same human subject. Samples in each set were subjected to the procedure as described in Example 4, for the purpose of detecting high-risk HPV subtypes in these samples. The results of the comparison are demonstrated in Table 6.

TABLE 6
Comparison of High-risk HPV tests using urine samples and
cervical exfoliated cell samples

| | | Cervical exfoliated cell samples | | |
|---|---|---|---|---|
| High-risk HPV | | Positive | Negative | Total |
| Urine | Positive | 8 | 0 | 8 |
| samples | Negative | 2 | 80 | 82 |
| | Total | 10 | 80 | 90 |

| | |
|---|---|
| Positive matching rate | 80.00% |
| Negative matching rate | 100.00% |
| Total matching rate | 97.78% |
| Kappa value | 0.877 |

The results indicate that by using the compositions and methods of the present disclosure, our test using urine samples achieved high sensitivity and specificity which are comparable to the tests using cervical exfoliated cell samples.

Thus, the presently disclosed compositions and methods for detecting HPV in urine samples provide a non-invasive, harmless, and painless way for encouraging and simplifying HPV test, compared to the traditional methods involving female cervical exfoliated cell samples or male urethral swab samples.

### Example 6: To Evaluate the Effectiveness of Urine HPV Test in Cervical Cancer Screening

The 1,381 subjects were selected from women who had been diagnosed as HPV positive or negative in the previous year in Shanxi Province, China. Urine samples and cervical exfoliated cells samples were collected for each subject respectively. Urine samples were tested with the urine HPV detection reagent in the invention, while cervical exfoliated cells were tested with HPV nucleic acid detection reagent (bohui-tech) by microfluidic chip method. If the test result of cervical exfoliated cells is positive, pathological confirmation is performed. Finally, the pathological results were used as the gold standard to compare the efficacy of urine HPV nucleic acid detection technology and microfluidized microchip HPV detection technology for cervical cancer screening. The results are shown in Table 7 and Table 8.

TABLE 7
The urine HPV test of the invention is used to evaluate the effect
of cervical cancer screening

| | | pathology | | |
|---|---|---|---|---|
| | | CIN ≥ 2 | CIN < 2 | total |
| urine | positive | 30 | 527 | 557 |
| HPV test | negative | 1 | 823 | 824 |
| | total | 31 | 1350 | 1381 |

| | |
|---|---|
| sensitivity | 96.77% |
| specificity | 60.96% |
| positive predictive value | 5.38% |
| negative predictive value | 99.9% |

TABLE 8
Microfluidized microchip HPV test was used to evaluate the
effectiveness of screening cervical cancer

| | | pathology | | |
|---|---|---|---|---|
| | | CIN ≥ 2 | CIN < 2 | total |
| Microfluidized | positive | 31 | 558 | 589 |
| microchip HPV | negative | 0 | 792 | 792 |
| test | total | 31 | 1350 | 1381 |

| | |
|---|---|
| sensitivity | 100.00% |
| specificity | 58.67% |
| positive predictive value | 5.26% |
| negative predictive value | 100.0% |

It can be seen from the above test results that the urine HPV detection technology in the invention is used for cervical cancer screening, and its effect is basically the same as that of the microfluidic chip HPV detection technology.

1. A combination of primer and probe for detecting and/or identifying a genotype of a human papillomavirus (HPV), wherein the combination of primer and probe comprises one or more groups of primer and probe selected from the groups consisting of:
(1) a forward primer comprising a polynucleotide sequence having at least 85%, 90%, 95%, or 100% identity to a sequence of SEQ ID NO: 1, a reverse primer comprising a polynucleotide sequence having at least 85%, 90%, 95%, or 100% identity to a sequence of SEQ ID NO: 2, and a probe comprising a polynucleotide sequence having at least 85%, 90%, 95%, or 100% identity to a sequence of SEQ ID NO: 37;
(2) a forward primer comprising a polynucleotide sequence having at least 85%, 90%, 95%, or 100% identity to a sequence of SEQ ID NO: 3, a reverse primer comprising a polynucleotide sequence having at least 85%, 90%, 95%, or 100% identity to a sequence of SEQ ID NO: 4, and a probe comprising a polynucleotide sequence having at least 85%, 90%, 95%, or 100% identity to a sequence of SEQ ID NO: 38;
(3) a forward primer comprising a polynucleotide sequence having at least 85%, 90%, 95%, or 100% identity to a sequence of SEQ ID NO: 5, a reverse primer comprising a polynucleotide sequence having at least 85%, 90%, 95%, or 100% identity to a sequence of SEQ ID NO: 6, and a probe comprising a polynucleotide sequence having at least 85%, 90%, 95%, or 100% identity to a sequence of SEQ ID NO: 39;
(4) a forward primer comprising a polynucleotide sequence having at least 85%, 90%, 95%, or 100% identity to a sequence of SEQ ID NO: 7, a reverse primer comprising a polynucleotide sequence having at least 85%, 90%, 95%, or 100% identity to a sequence of SEQ ID NO: 8, and a probe comprising a polynucleotide sequence having at least 85%, 90%, 95%, or 100% identity to a sequence of SEQ ID NO: 39;
(5) a forward primer comprising a polynucleotide sequence having at least 85%, 90%, 95%, or 100% identity to a sequence of SEQ ID NO: 9, a reverse primer comprising a polynucleotide sequence having at least 85%, 90%, 95%, or 100% identity to a sequence of SEQ ID NO: 10, and a probe comprising a polynucleotide sequence having at least 85%, 90%, 95%, or 100% identity to a sequence of SEQ ID NO: 39;
(6) a forward primer comprising a polynucleotide sequence having at least 85%, 90%, 95%, or 100% identity to a sequence of SEQ ID NO: 11, a reverse primer comprising a polynucleotide sequence having at least 85%, 90%, 95%, or 100% identity to a sequence of SEQ ID NO: 12, and a probe comprising a polynucleotide sequence having at least 85%, 90%, 95%, or 100% identity to a sequence of SEQ ID NO: 40;
(7) a forward primer comprising a polynucleotide sequence having at least 85%, 90%, 95%, or 100% identity to a sequence of SEQ ID NO: 13, a reverse primer comprising a polynucleotide sequence having at least 85%, 90%, 95%, or 100% identity to a sequence of SEQ ID NO: 14, and a probe comprising a polynucleotide sequence having at least 85%, 90%, 95%, or 100% identity to a sequence of SEQ ID NO: 41;
(8) a forward primer comprising a polynucleotide sequence having at least 85%, 90%, 95%, or 100% identity to a sequence of SEQ ID NO: 15, a reverse primer comprising a polynucleotide sequence having at least 85%, 90%, 95%, or 100% identity to a sequence of SEQ ID NO: 16, and a probe comprising a polynucleotide sequence having at least 85%, 90%, 95%, or 100% identity to a sequence of SEQ ID NO: 42;
(9) a forward primer comprising a polynucleotide sequence having at least 85%, 90%, 95%, or 100% identity to a sequence of SEQ ID NO: 17, a reverse primer comprising a polynucleotide sequence having at least 85%, 90%, 95%, or 100% identity to a sequence of SEQ ID NO: 18, and a probe comprising a polynucleotide sequence having at least 85%, 90%, 95%, or 100% identity to a sequence of SEQ ID NO: 42;
(10) a forward primer comprising a polynucleotide sequence having at least 85%, 90%, 95%, or 100% identity to a sequence of SEQ ID NO: 19, a reverse primer comprising a polynucleotide sequence having at least 85%, 90%, 95%, or 100% identity to a sequence of SEQ ID NO: 20, and a probe comprising a polynucleotide sequence having at least 85%, 90%, 95%, or 100% identity to a sequence of SEQ ID NO: 41;
(11) a forward primer comprising a polynucleotide sequence having at least 85%, 90%, 95%, or 100% identity to a sequence of SEQ ID NO: 21, a reverse primer comprising a polynucleotide sequence having at least 85%, 90%, 95%, or 100% identity to a sequence of SEQ ID NO: 22, and a probe comprising a polynucleotide sequence having at least 85%, 90%, 95%, or 100% identity to a sequence of SEQ ID NO: 39;
(12) a forward primer comprising a polynucleotide sequence having at least 85%, 90%, 95%, or 100% identity to a sequence of SEQ ID NO: 23, a reverse primer comprising a polynucleotide sequence having at least 85%, 90%, 95%, or 100% identity to a sequence of SEQ ID NO: 24, and a probe comprising a polynucleotide sequence having at least 85%, 90%, 95%, or 100% identity to a sequence of SEQ ID NO: 40;
(13) a forward primer comprising a polynucleotide sequence having at least 85%, 90%, 95%, or 100% identity to a sequence of SEQ ID NO: 25, a reverse primer comprising a polynucleotide sequence having at least 85%, 90%, 95%, or 100% identity to a sequence of SEQ ID NO: 26, and a probe comprising a polynucleotide sequence having at least 85%, 90%, 95%, or 100% identity to a sequence of SEQ ID NO: 41;
(14) a forward primer comprising a polynucleotide sequence having at least 85%, 90%, 95%, or 100% identity to a sequence of SEQ ID NO: 27, a reverse primer comprising a polynucleotide sequence having at least 85%, 90%, 95%, or 100% identity to a sequence of SEQ ID NO: 28, and a probe comprising a polynucleotide sequence having at least 85%, 90%, 95%, or 100% identity to a sequence of SEQ ID NO: 40;
(15) a forward primer comprising a polynucleotide sequence having at least 85%, 90%, 95%, or 100% identity to a sequence of SEQ ID NO: 29, a reverse primer comprising a polynucleotide sequence having at least 85%, 90%, 95%, or 100% identity to a sequence of SEQ ID NO: 30, and a probe comprising a polynucleotide sequence having at least 85%, 90%, 95%, or 100% identity to a sequence of SEQ ID NO: 40;
(16) a forward primer comprising a polynucleotide sequence having at least 85%, 90%, 95%, or 100% identity to a sequence of SEQ ID NO: 33, a reverse primer comprising a polynucleotide sequence having at least 85%, 90%, 95%, or 100% identity to a sequence of SEQ ID NO: 34, and a probe comprising a polynucleotide sequence having at least 85%, 90%, 95%, or 100% identity to a sequence of SEQ ID NO: 44;
(17) a forward primer comprising a polynucleotide sequence having at least 85%, 90%, 95%, or 100% identity to a sequence of SEQ ID NO: 35, a reverse primer comprising a polynucleotide sequence having at least 85%, 90%, 95%, or 100% identity to a sequence of SEQ ID NO: 36, and a probe comprising a polynucleotide sequence having at least 85%, 90%, 95%, or 100% identity to a sequence of SEQ ID NO: 45; and any combination thereof.

2. The combination of primer and probe of claim 1, wherein the combination of primer and probe comprises the primers and probes in (1) and (2).

3. The combination of primer and probe of claim 1, wherein the combination of primer and probe comprises the primers and probes in (3), (4), (5), and (11).

4. The combination of primer and probe of claim 1, wherein the combination of primer and probe comprises the primers and probes in (6), (12), (14) and (15).

5. The combination of primer and probe of claim 1, wherein the combination of primer and probe comprises the primers and probes in (7), (10), and (13).

6. The combination of primer and probe of claim 1, wherein the combination of primer and probe comprises the primers and probes in (8) and (9).

7. The combination of primer and probe of claim 1, wherein the combination of primer and probe comprises: at least one group of primers and probes in (1) and (2) and at least one group of primers and probes in (3), (4), (5), and (11).

8. The combination of primer and probe of claim 1, wherein the combination of primer and probe comprises: at least one group of primers and probes in (1) and (2) and at least one group of primers and probes in (6), (12), (14) and (15).

9. The combination of primer and probe of claim 1, wherein the combination of primer and probe comprises: at least one group of primers and probes in (1) and (2) and at least one group of primers and probes in (7), (10), and (13).

10. The combination of primer and probe of claim 1, wherein the combination of primer and probe comprises: at least one group of primers and probes in (1) and (2) and at least one group of primers and probes in (8) and (9).

11. The combination of primer and probe of claim 1, wherein the combination of primer and probe comprises: at least one group of primers and probes in (1) and (2), at least one group of primers and probes in (3), (4), (5), and (11), and at least one group of primers and probes in (6), (12), (14) and (15).

12. The combination of primer and probe of claim 1, wherein the combination of primer and probe comprises: at least one group of primers and probes in (1) and (2), at least one group of primers and probes in (3), (4), (5), and (11), and at least one group of primers and probes in (7), (10), and (13).

13. The combination of primer and probe of claim 1, wherein the combination of primer and probe comprises: at least one group of primers and probes in (1) and (2), at least one group of primers and probes in (3), (4), (5), and (11), and at least one group of primers and probes in (8) and (9).

14. The combination of primer and probe of claim 1, wherein the combination of primer and probe comprises: at least one group of primers and probes in (1) and (2), at least one group of primers and probes in (6), (12), (14) and (15), and at least one group of primers and probes in (7), (10), and (13).

15. The combination of primer and probe of claim 1, wherein the combination of primer and probe comprises: at least one group of primers and probes in (1) and (2), at least one group of primers and probes in (6), (12), (14) and (15), and at least one group of primers and probes in (8) and (9).

16. The combination of primer and probe of claim 1, wherein the combination of primer and probe comprises: at least one group of primers and probes in (1) and (2), at least one group of primers and probes in (7), (10), and (13), and at least one group of primers and probes in (8) and (9).

17. The combination of primer and probe of claim 1, wherein the combination of primer and probe comprises: at least one group of primers and probes in (1) and (2), at least one group of primers and probes in (3), (4), (5), and (11), at least one group of primers and probes in (6), (12), (14) and (15), and at least one group of primers and probes in (7), (10), and (13).

18. The combination of primer and probe of claim 1, wherein the combination of primer and probe comprises: at least one group of primers and probes in (1) and (2), at least one group of primers and probes in (3), (4), (5), and (11), at least one group of primers and probes in (6), (12), (14) and (15), and at least one group of primers and probes in (8) and (9).

19. The combination of primer and probe of claim 1, wherein the combination of primer and probe comprises: at least one group of primers and probes in (1) and (2), at least one group of primers and probes in (3), (4), (5), and (11), and at least one group of primers and probes in (7), (10), and (13), and at least one group of primers and probes in (8) and (9).

20. The combination of primer and probe of claim 1, wherein the combination of primer and probe comprises: at least one group of primers and probes in (1) and (2), at least one group of primers and probes in (6), (12), (14) and (15), and at least one group of primers and probes in (7), (10), and (13), and at least one group of primers and probes in (8) and (9).

21. The combination of primer and probe of any one of claims 2 to 20, wherein the combination of primer and probe further comprises the group of primers and probes in (16) and/or (17).

22. The combination of primer and probe of claim 1, wherein the combination of primer and probe consists of the group of primers and probes in (1) to (15).

23. The combination of primer and probe of claim 1, wherein the combination of primer and probe consists of the group of primers and probes in (1) to (17).

24. The combination of primer and probe of any one of claims 1 to 23, wherein the combination of primer and probe further comprises a group of primer and probe for a reference control gene.

25. The combination of primer and probe of claim 24, wherein the reference control gene is β-Actin (ACTB), wherein the primers for the ACTB gene are SEQ ID NOs: 31 and 32, and the probe for the ACTB gene is SEQ ID NO: 43.

26. The combination of primer and probe of any one of claims 1 to 25, wherein each probe has a fluorescent dye attached to 5′ end thereof.

27. The combination of primer and probe of claim 26, wherein the fluorescent dye is selected from the group consisting of FAM (fluorescein), TET, JOE, VIC, HEX, ROX, TAMRA, Cy3, cy3.5, Cy5, Cy5.5, OregonGreen™, CALRed™, Red640, Texas Red, LighterCycler®Cyan500, LighterCycler®, Red610, a biotin binding material, Alexa 647, Alexa 555, 5-(2-aminoethyl)amino-1-naphthalene sulfonic acid (EDANS), tetramethyl rhodamine (TMR), tetramethyl rhodamine isocyanate (TMRITC), fluorescein isocyanate (FITC), and χ-rhodamine.

28. The combination of primer and probe of claim 26, wherein the fluorescent dye on the probes in (3), (4), (5), and (11) is the same dye or different dyes having about the same or different emission wavelength.

29. The combination of primer and probe of claim 26, wherein the fluorescent dye on the probes in (6), (12), (14) and (15) is the same dye, or different dyes having about the same or different emission wavelength.

30. The combination of primer and probe of claim 26, wherein the fluorescent dye on the probes in (7), (10), and (13) is the same dye, or different dyes having about the same or different emission wavelength.

31. The combination of primer and probe of claim 26, wherein the fluorescent dye on the probes in (8) and (9) is the same dye, or different dyes having about the same or different emission wavelength.

32. The combination of primer and probe of claim 22, wherein each probe has a fluorescent dye attached to 5′ end thereof, and wherein:
(i) the probe in (1) has a first dye;
(ii) the probe in (2) has a second dye;
(iii) the probes in (3), (4), (5), and (11) have a third dye;
(iv) the probes in (6), (12), (14) and (15) have a fourth dye;
(v) the probes in (7), (10), and (13) have a fifth dye; and
(vi) the probes in (8) and (9) have a sixth dye, wherein
the dye in (iii) to (vi) are the same dye, but different from the dye in (i) and (ii).

33. The combination of primer and probe of claim 23, wherein each probe has a fluorescent dye attached to 5′ end thereof, and wherein:
(i) the probe in (1) has a first dye;
(ii) the probe in (2) has a second dye;
(iii) the probes in (3), (4), (5), and (11) have a third dye;
(iv) the probes in (6), (12), (14) and (15) have a fourth dye;
(v) the probes in (7), (10), and (13) have a fifth dye;
(vi) the probes in (8) and (9) have a sixth dye,
(vii) the probe in (16) has a seventh dye; and
(viii) the probe in (17) has an eighth dye; wherein
the dye in (iii) to (vi) are the same dye, but different from the dye in (i), (ii), (vii) and (viii).

34. The combination of primer and probe of claims 32 and 33, wherein the combination of primers and probes further comprises a group of primers and probe for a reference control gene, wherein the probe for the reference control gene also has a fluorescent dye attached to 5′ end thereof, and the fluorescent dye for the control gene is different from other dyes in the combination.

35. The combination of primer and probe of any one of claims 1 to 23, wherein each probe has a fluorescent quencher attached to 3′ end thereof.

36. The combination of primer and probe of claim 35, wherein the fluorescent quencher is selected from the group consisting of DDQ-I, DDQ-II, Dabcyl, Eclipse, Iowa Black FQ, Iowa Black RQ, BHQ-1, BHQ-2, BHQ-3, QSY-7, QSY-9, and QSY-21.

37. The combination of primer and probe of claim 36, wherein:
the probe in (1) comprises a Cy5 fluorescent dye attached to its 5′ end, and a BHQ-2 fluorescent quencher attached to its 3′ end;
the probe in (2) comprises a FAM fluorescent dye attached to its 5′ end, and a BHQ-1 fluorescent quencher attached to its 3′ end;
the probes in (3) to (15) comprise a VIC fluorescent dye attached to their 5′ ends, and a MGBNFQ fluorescent quencher attached to their 3′ ends; and
the probes in (16) and (17) comprises a FAM fluorescent dye attached to their 5′ ends, and a BHQ-1 fluorescent quencher attached to their 3′ ends.

38. The combination of primer and probe of claim 35, wherein:
the probe in (1) comprises a Cy5 fluorescent dye attached to its 5′ end, and a BHQ-2 fluorescent quencher attached to its 3′ end;
the probe in (2) comprises a FAM fluorescent dye attached to its 5′ end, and a BHQ-1 fluorescent quencher attached to its 3′ end;
the probes in (3) to (15) comprise a VIC fluorescent dye attached to their 5′ ends, and a MGBNFQ fluorescent quencher attached to their 3′ ends;
the probes in (16) and (17) comprises a FAM fluorescent dye attached to their 5′ ends, and a BHQ-1 fluorescent quencher attached to their 3′ ends, and
the probe for the reference control gene comprises a ROX fluorescent dye attached to its 5′ end, and a BHQ-2 fluorescent quencher attached to its 3′ end.

39. A composition comprising the combination of primer and probe of any one of claims 1 to 38.

40. A DNA chip for testing HPV genotypes, comprising one or more polynucleotides selected from SEQ ID NOs: 1 to 45.

41. A kit for detecting and/or identifying the genotype of a human papillomavirus (HPV) in a biological sample, comprising a combination of primer and probe in any one of claims 1 to 38.

42. The kit of claim 41, wherein the biological sample is collected from a human subject.

43. The kit of claim 42, wherein the biological sample comprises urine of the human subject.

44. The kit of claim 41, wherein the kit further comprises reagents for isolating DNA from the biological sample, and/or further comprises a negative and/or positive control DNA template.

45. The kit of claim 44, wherein the reagents for isolating DNA from the biological sample comprises: a lysis solution, magnetic nanoparticles, a protease, a first washing buffer, a second washing buffer, an elution buffer, or any combination thereof.

46. The kit of claim 45, wherein the lysis solution comprises guanidinium isothiocyanate, Triton X 100, Tris-HCl, EDTA, and isopropanol.

47. The kit of claim 46, wherein the guanidinium isothiocyanate has a concentration of about 1 to 2 M.

48. The kit of claim 46, wherein the Triton X 100 has a concentration of about 1 to 2%.

49. The kit of claim 46, wherein the Tris-HCl has a concentration of about 5 to 10 mM, wherein the lysis solution has a pH of about 6-7.

50. The kit of claim 46, wherein the EDTA has a concentration of about 3 to 5 mM.

51. The kit of claim 46, wherein the isopropanol has a volume of about 50% to 80% (v/v) of the lysis solution.

52. The kit of claim 45, wherein the magnetic nanoparticles have an inner core layer and an outer shell layer, wherein the inner core layer is composed of core-shell type magnetic nanoparticles, wherein the outer shell layer is composed of SiO₂.

53. The kit of claim 51, wherein the magnetic nanoparticles have a diameter of about 100 to 1000 nm, and a concentration of about 50 mg/ml.

54. The kit of claim 45, wherein the first washing buffer comprises guanidinium isothiocyanate, Tris-HCl, NaCl, and ethanol.

55. The kit of claim 54, wherein the guanidinium isothiocyanate has a concentration of about 50 mM.

56. The kit of claim 54, wherein the Tris-HCl has a concentration of about 20 to 50 mM,

57. The kit of claim 46, wherein the first washing buffer has a pH of about 5.0.

58. The kit of claim 54, wherein the NaCl has a concentration of about 50 to 200 mM.

59. The kit of claim 54, wherein the ethanol has concentration of about 40% to 60% (v/v).

60. The kit of claim 45, wherein the second washing buffer comprises Tris-HCl and ethanol.

61. The kit of claim 60, wherein the Tri-HCl in the second washing buffer has a concentration of about 10 to 50 mM, and the second washing buffer has a pH of about 6.0.

62. The kit of claim 60, wherein the ethanol has a concentration of about 70% to 80% (v/v).

63. The kit of claim 45, wherein the elution buffer is a Tris-EDTA buffer having a pH of about 8.0.

64. The kit of claim 45, wherein the protease is protease K.

65. The kit of claim 64, wherein the protease K has a concentration of about 10 to 20 mg/ml.

66. A method for detecting and/or identifying the genotype of a human papillomavirus (HPV) in a biological sample obtained from a subject in need thereof, comprising:
(a) obtaining DNA from the biological sample;
(b) amplifying the DNA by a fluorescent PCR using a combination of primer and probe in any one of claims 1 to 38; and
(c) determining the presence or absence of DNA of one or more HPV subtype in the biological sample based on the results of the fluorescent PCR.

67. A method for detecting and/or identifying the genotype of a human papillomavirus (HPV) in a biological sample obtained from a subject in need thereof, comprising:
(a) obtaining DNA from the biological sample;
(b) amplifying the DNA by a fluorescent PCR using the kit of claim 31; and
(c) determining the presence or absence of DNA of one or more HPV subtype in the biological sample based on the results of the fluorescent PCR.

68. A method for detecting and/or identifying the genotype of a human papillomavirus (HPV) in a biological sample obtained from a subject in need thereof, comprising:
(a) extracting DNA from the biological sample and amplifying the DNA by a fluorescent PCR using the kit of any one of claims 42 to 63; and
(b) determining the presence or absence of DNA of one or more HPV subtype in the biological sample based on the results of the fluorescent PCR.

69. The method of any one of claims 66 to 68, wherein the method comprises detecting and/or identifying the presence or absence of DNA of at least seven HPV subtypes in the biological sample.

70. The method of any one of claims 66 to 68, wherein the method comprises detecting and/or identifying the presence or absence of DNA of 14 high-risk HPV subtypes in the biological sample through a single test tube, wherein the high-risk HPV subtypes are HPV16, HPV18, HPV31, HPV33, HPV35, HPV39, HPV45, HPV51, HPV52, HPV56, HPV58, HPV59, HPV66, and HPV68.

71. The method of any one of claims 66 to 68, wherein the method comprises detecting and/or identifying the presence or absence of DNA of 14 high-risk HPV subtypes and at least one low-risk HPV subtype in the biological sample through a single test tube, wherein the high-risk HPV subtypes are HPV16, HPV18, HPV31, HPV33, HPV35, HPV39, HPV45, HPV51, HPV52, HPV56, HPV58, HPV59, HPV66, and HPV68, and the at least one low-risk HPV subtype is HPV6 or HPV11.

72. The method of claim 70, wherein the biological sample is a smear of the cervix, a fresh tissue sample, a fixed tissue sample, a cross-sectional specimen of a tissue sample, a urine sample, a sample comprising exfoliated cells, a peripheral blood sample, a penis swab, or other body fluid.

73. The method of claim 72, wherein the sample is a urine sample.

74. Use of a combination of primer and probe of any one of claims 1 to 38 for detecting and/or identifying the genotype of a human papillomavirus (HPV).

75. A method for treating a condition associated with human papillomavirus (HPV) in a subject in need thereof, comprising:
(1) detecting and/or identifying the genotype of a human papillomavirus (HPV) in a biological sample obtained from a subject in need thereof, comprising:
(a) amplifying DNA extracted from the biological sample by a fluorescent PCR using a combination of primers and probes of any one of claims 1 to 38; and
(b) determining the presence or absence of DNA of one or more HPV subtype in the biological sample based on the results of the fluorescent PCR, and
(2) treating the subject with a pharmaceutical composition and/or a medical procedure according to the results in step (1).

76. The method of claim 75, wherein the condition is a precancerous lesions caused by HPV.

77. The method of claim 75, wherein the pharmaceutical composition comprises an antiviral agent.

78. A method for vaccinating a human subject in need thereof, comprising:
(1) detecting and/or identifying the genotype of a human papillomavirus (HPV) in a biological sample obtained from the human subject in need thereof before and/or after the human subject is vaccinated, comprising:
(a) amplifying DNA extracted from the biological sample by a fluorescent PCR using a combination of primers and probes of any one of claims 1 to 38; and
(b) determining the presence or absence of DNA of one or more HPV subtype in the biological sample based on the results of the fluorescent PCR, and
(2) vaccinating the subject with a composition targeting selected HPVs based on the results in step (1).

79. A method for evaluating vaccination efficacy in a human subject in need thereof, comprising:
(1) detecting and/or identifying the genotype of a human papillomavirus (HPV) in a biological sample obtained from the human subject in need thereof after, or before and after the human subject is vaccinated, comprising:
(a) amplifying DNA extracted from the biological sample by a fluorescent PCR using a combination of primers and probes of any one of claims 1 to 38, after, or before and after the human subject is vaccinated; and
(b) determining the presence or absence of DNA of one or more HPV subtype in the biological sample based on the results of the fluorescent PCR, and
(2) vaccinating the subject with a composition targeting selected HPVs;
(3) determining vaccination efficacy based on the results in step (1).

80. The kit of claim 41, which further comprises reagents for testing for DNA amplification of 14 high-risk types of HPV.

81. The kit of claim 80, which is used for 14 high-risk HPV DNA amplification tests, further comprises HPV qPCR mixture, Taq enzyme, positive control, and negative control.

82. The kit of claim 81, the HPV qPCR mixture further comprises primer and probe compositions in claims 22 and 24, PCR buffer, dNTP, MgCl₂, PCR additive, and deionized water.

83. The kit of claim 82, the primer and probe composition has a primer and probe concentration of 0.1˜1.2 M, and the primer and probe combination includes all primer and probe combinations in (1)˜(15) as well as reference control gene primers and probes (SEQ ID NOs: 31, 32 and 43).

84. The kit of claim 82, the PCR buffer comprises 10˜30 mM Tris-HCl buffer and 30˜70 mM KCl, the preferred Tris-HCl buffer concentration is 20.5 mM and the preferred KCl concentration is approximately 51 mM.

85. The kit of claim 82, wherein the dNTP concentration is 0.15 mM˜0.3 mM and the preferred dNTP concentration is approximately 0.25 mM.

86. The kit of claim 82, the concentration of MgCl₂ is 1.5 mM˜4 mM, and the preferred concentration of MgCl₂ is about 3.0 mM.

87. The kit of claim 82, the PCR additive comprises about 0.1-1 mg/ml BSA, 0.2%-2% (V/V) formamide, 0.2 mM˜2 mM spermidine, 10 mM˜30 mM tetramethylammonium chloride, 0.01 mM˜0.1 mM DTT, 0.2%-2% 2-pyrrolidone, preferably, the PCR additive comprises about 0.64 mg/ml BSA, about 1% (VN) formamide, about 1 mM spermidine, about 21 mM tetramethylammonium chloride, about 0.064 mM DTT, about 1% (VN) 2-pyrrolidone.

88. The kit of claim 81, in which the Taq enzyme concentration is 1˜6U/μl, and the preferred Taq enzyme is Platinum Taq DNA Polymerase with a concentration of 4U/μl.

89. The kit of claim 81, the negative control is the urine or its DNA of adults with high risk HPV DNA negative is diluted by 1˜1000 times, and the optimal dilution is about 100 times.

90. The kit of claim 81, wherein the positive control is a plasmid containing high-risk HPV L1 gene with the final concentration of 10-10⁵ copies/μl using the negative control as the diluent, and the high-risk HPV L1 gene type may be one or more of the 14 high-risk HPV types described in the present invention. Preferably, the positive control contains L1 plasmids of HPV16, HPV18 and hpv45, and their final concentration is 10³ copies/μL.

91. A kit for the detection and/or recognition of human papillomavirus (HPV) genotypes in biological samples and for the guidance and efficacy evaluation of HPV vaccine.

92. The kit of claim 91, the kit further comprises reagents for DNA amplification detection of 16 types of HPV.

93. The kit of claim 92, wherein the reagents for the amplification and detection of 16 types of HPV DNA further include: HPV qPCR mixture I, HPV qPCR mixture II, Taq enzyme, positive control and negative control.

94. The kit of claim 93, wherein HPV qPCR mixture I further comprises primer and probe composition, PCR buffer, dNTP, MgCl₂, PCR additive and deionized water in claims 23 and 24.

95. The kit of claim 94, wherein the primer and probe concentration in the primer and probe composition is 0.1-1.2 μM, and the primer and probe combination includes all the primer and probe combinations in (1), (2), (5), (6), (8), (10), (12), (13), (14), (15), and the internal gene primers and probes (SEQ ID Nos: 31, 32 and 43).

96. The kit of claim 94, wherein the PCR buffer comprises 10-30 mM Tris-HCl buffer and 30-70 mM KCl, the preferred Tris-HCl buffer concentration is 20.5 mM, and the preferred KCl concentration is about 51 mM.

97. The kit of claim 94, wherein the dNTP concentration is 0.15 mM˜0.3 mM, and the preferred dNTP concentration is about 0.25 mM.

98. The kit of claim 94, wherein the concentration of MgCl₂ is 1.5 mM˜4 mM, and the preferred concentration of MgCl₂ is about 3.0 mM.

99. The kit of claim 94, wherein the PCR additive comprises: about 0.1-1 mg/ml BSA, 0.2%-2% (VN) formamide, 0.2 mM˜2 mM spermidine, 10 mM˜30 mM tetramethylammonium chloride, 0.01 mM˜0.1 mM DTT, 0.2%-2% 2-pyrrolidone, preferably, the PCR additive includes about 0.64 mg/ml BSA, about 1% (VN) formamide, about 1 mM spermidine, about 21 mM tetramethylammonium chloride, about 0.064 mM DTT, about 1% (VN) 2-pyrrolidone.

100. The kit of claim 93, wherein HPV qPCR mixture II further comprises primer and probe composition in claims 23 and 24, PCR buffer, dNTP, MgCl₂, PCR additive and deionized water.

101. The kit of claim 100, wherein the primer probe concentration in the primer probe composition is 0.1-1.2 μm, and the primer and probe combination includes all the primer probe combinations in (3), (4), (7), (9), (11), (16), (17) and the reference control gene primers and probes (SEQ ID NOs: 31, 32 and 43).

102. The kit of claim 100, wherein the PCR buffer comprises 10-30 mM Tris-HCl buffer and 30-70 mM KCL, the preferred Tris-HCl buffer concentration is 20.5 mM, and the preferred KCl concentration is about 51 mM.

103. The kit of claim 100, wherein the dNTP concentration is 0.15 mM˜0.3 mM, and the preferred dNTP concentration is about 0.25 mM.

104. The kit of claim 100, wherein the concentration of MgCl₂ is 1.5 mM˜4 mM, and the preferred concentration of MgCl₂ is about 3.0 mM.

105. The kit of claim 100, wherein the PCR additive comprises about 0.1-1 mg/ml BSA, 0.2%-2% (VN) formamide, 0.2 mM˜2 mM spermidine, 10 mM˜30 mM tetramethylammonium chloride, 0.01 mM˜0.1 mM DTT, 0.2%-2% 2-pyrrolidone, preferably, the PCR additive comprises about 0.64 mg/ml BSA, about 1% (V/V) formamide, about 1 mM spermidine, about 21 mM tetramethylammonium chloride, about 0.064 mM DTT, about 1% (V/V) 2-pyrrolidone.

106. A primer, comprising an oligonucleotide sequence having at least 85%, 90%, 95%, or 100% identity to any one of SEQ ID NOs: 1-36, wherein the primer has less than 100, 90, 80, 70, 60, 50, 40, 30, or 20 nucleotides.

107. A pair of primers, comprising a forward primer and a reverse primer, each having at least 85%, 90%, 95%, or 100% identity to any one of SEQ ID NOs: 1-36 and each having less than 100, 90, 80, 70, 60, 50, 40, 30, or 20 nucleotides, wherein the forward and reverse primers are selected from the groups consisting of: SEQ ID NOs: 1-2, 3-4, 5-6, 7-8, 9-10, 11-12, 13-14, 15-16, 17-18, 19-20, 21-22, 23-24, 25-26, 27-28, 29-30, 31-32, 33-34, 35-36, and any combination thereof.

108. A probe, comprising a fluorescent dye and an oligonucleotide, wherein the oligonucleotide comprises a sequence having at least 85%, 90%, 95%, or 100% identity to any one of SEQ ID NOs: 37-45, wherein the oligonucleotide has less than 100, 90, 80, 70, 60, 50, 40, 30, or 20 nucleotides.

109. The probe of claim 108, wherein the fluorescent dye is attached to 5′ end of the probe.

110. The probe of claim 108 or 109, wherein the fluorescent dye is selected from the group consisting of FAM (fluorescein), TET, JOE, VIC, HEX, ROX, TAMRA, Cy3, cy3.5, Cy5, Cy5.5, OregonGreen™, CALRed™, Red640, Texas Red, LighterCycler®Cyan500, LighterCycler®, Red610, a biotin binding material, Alexa 647, Alexa 555, 5-(2-aminoethyl)amino-1-naphthalene sulfonic acid (EDANS), tetramethyl rhodamine (TMR), tetramethyl rhodamine isocyanate (TMRITC), fluorescein isocyanate (FITC), and χ-rhodamine.

111. The probe of any one of claims 108-110, further comprising a fluorescent quencher.

112. The probe of claim 111, wherein the fluorescent quencher is attached to 3′ end of the probe.

113. The probe of claim 111 or 112, wherein the fluorescent quencher is selected from the group consisting of DDQ-I, DDQ-II, Dabcyl, Eclipse, Iowa Black FQ, Iowa Black RQ, BHQ-1, BHQ-2, BHQ-3, QSY-7, QSY-9, and QSY-21.

114. A kit for detecting and/or identifying the genotype of a human papillomavirus (HPV) in a biological sample, comprising one or more primers of any one of claim 106 or 107, and a one or more probes of any one of claims 108-113.

115. The kit of claim 114, comprising at least 2, 4, 6, 8, 10, 12, 14, 16, 18, 20, 22, 24, 26, 28, 30, 32, 34, or 36 primers selected from any one of claim 106 or 107.

116. The kit of claim 114 or 115, comprising at least 2, 3, 4, 5, 6, 7, 8, or 9 probes selected from any one of claim 108-113.

117. The kit of any one of claims 114-116, further comprising a lysis solution, magnetic nanoparticles, a protease, a first washing buffer, a second washing buffer, an elution buffer, or any combination thereof.

118. A method for detecting and/or identifying the genotype of a human papillomavirus (HPV) in a urine sample obtained from a subject in need thereof, comprising:
(a) obtaining DNA from the urine sample;
(b) amplifying the DNA by a fluorescent PCR using one or more primers of any one of claim 106 or 107, and a one or more probes of any one of claims 108-113; and
(c) determining the presence or absence of DNA of one or more HPV subtype in the biological sample based on the results of the fluorescent PCR.


SEQUENCE LISTING (48 sequences)

SEQ ID NO: 1 - DNA, 29 bp, Human papillomavirus type 16
gattgtccaccattagagttaataaacac

SEQ ID NO: 2 - DNA, 26 bp, Human papillomavirus type 16
gcctgtaatgtagtaaagtccatagc

SEQ ID NO: 3 - DNA, 20 bp, Human papillomavirus type 18
acaggtatgcgtgcttcacc

SEQ ID NO: 4 - DNA, 26 bp, Human papillomavirus type 18
tttattaaacaactgggagtcagagg

SEQ ID NO: 5 - DNA, 22 bp, Human papillomavirus type 31
ggtggtcctggcactgataata

SEQ ID NO: 6 - DNA, 22 bp, Human papillomavirus type 31
ctccaataggtggtttgcaacc

SEQ ID NO: 7 - DNA, 21 bp, Human papillomavirus type 33
tggacaaccgggtgctgataa

SEQ ID NO: 8 - DNA, 21 bp, Human papillomavirus type 33
ccctgttggaggcttacatcc

SEQ ID NO: 9 - DNA, 29 bp, Human papillomavirus type 35
aatatgttggtaactctggtacagataac

SEQ ID NO: 10 - DNA, 22 bp, Human papillomavirus type 35
gtgtgccttttccccaatgttc

SEQ ID NO: 11 - DNA, 20 bp, Human papillomavirus type 39
gtggtcgcaagcaggacatt

SEQ ID NO: 12 - DNA, 27 bp, Human papillomavirus type 39
ctgaatttattaggatcgggcaatgtc

SEQ ID NO: 13 - DNA, 29 bp, Human papillomavirus type 45
aggtacatatgatcctactaagtttaagc

SEQ ID NO: 14 - DNA, 25 bp, Human papillomavirus type 45
acctctgcagttaaagtaatagtgc

SEQ ID NO: 15 - DNA, 22 bp, Human papillomavirus type 51
tcacgcatagcaaatggcaatg

SEQ ID NO: 16 - DNA, 21 bp, Human papillomavirus type 51
ccagtgttccccaataggtgg

SEQ ID NO: 17 - DNA, 28 bp, Human papillomavirus type 52
ggtaaacctggtatagataatagggaat

SEQ ID NO: 18 - DNA, 27 bp, Human papillomavirus type 52
tcctgaattattattacaaggggttcc

SEQ ID NO: 19 - DNA, 29 bp, Human papillomavirus type 56
gctacagaacagttaagtaaatatgatgc

SEQ ID NO: 20 - DNA, 29 bp, Human papillomavirus type 56
atattatgtaaatatgccataacctctgc

SEQ ID NO: 21 - DNA, 22 bp, Human papillomavirus type 58
gcagggtctgataacagggaat

SEQ ID NO: 22 - DNA, 22 bp, Human papillomavirus type 58
gctcaccagtgggaggtttaca

SEQ ID NO: 23 - DNA, 24 bp, Human papillomavirus type 59
aaggtggtaatggtagacaggatg

SEQ ID NO: 24 - DNA, 28 bp, Human papillomavirus type 59
cgttgagagttaggatcatatactgtgt

SEQ ID NO: 25 - DNA, 28 bp, Human papillomavirus type 66
accagaagtaccaacatgactattaatg

SEQ ID NO: 26 - DNA, 29 bp, Human papillomavirus type 66
aggttattttacaaagttgaaacacaaac

SEQ ID NO: 27 - DNA, 29 bp, Human papillomavirus type 68a
ctatgctggtacatctaggttattaactg

SEQ ID NO: 28 - DNA, 26 bp, Human papillomavirus type 68a
atcaggtaaggtaaccctaaacactc

SEQ ID NO: 29 - DNA, 30 bp, Human papillomavirus type 68b
attactatgctggtacatctaggttattaa

SEQ ID NO: 30 - DNA, 29 bp, Human papillomavirus type 68b
gactaaatttattaggatcaggtagggaa

SEQ ID NO: 31 - DNA, 22 bp, Homo sapiens
aggcatcctcaccctgaagtac

SEQ ID NO: 32 - DNA, 22 bp, Homo sapiens
acacgcagctcattgtagaagg

SEQ ID NO: 33 - DNA, 25 bp, Human papillomavirus type 6
gggtaatcaactgtttgttactgtg

SEQ ID NO: 34 - DNA, 27 bp, Human papillomavirus type 6
catgacgcatgtactctttataatcag

SEQ ID NO: 35 - DNA, 26 bp, Human papillomavirus type 11
tgcattacctgattcatctctgtttg

SEQ ID NO: 36 - DNA, 27 bp, Human papillomavirus type 11
catcatatttgtttagcaatggatgcc

SEQ ID NO: 37 - DNA, 30 bp, Human papillomavirus type 16
tgaccacgacctacctcaacacctacacag

SEQ ID NO: 38 - DNA, 30 bp, Human papillomavirus type 18
tgtgtgtattctccctctccaagtggctct

SEQ ID NO: 39 - DNA, 20 bp, Unknown
  Human papillomavirus types 31, 33, 35 and 58
atggattataaacaaacaca

SEQ ID NO: 40 - DNA, 17 bp, Unknown
  Human papillomavirus types 39, 59 and 68
tattgatatgcagacac

SEQ ID NO: 41 - DNA, 17 bp, Unknown
  Human papillomavirus types 45, 56 and 66
catgtggaggaatatga

SEQ ID NO: 42 - DNA, 14 bp, Unknown
  Human papillomavirus types 51 and 52
cagactcagttatg

SEQ ID NO: 43 - DNA, 25 bp, Homo sapiens
cgagcacggcatcgtcaccaactgg

SEQ ID NO: 44 - DNA, 30 bp, Human papillomavirus type 6
agataccacacgcagtaccaacatgacatt

SEQ ID NO: 45 - DNA, 29 bp, Human papillomavirus type 11
ccactacacagcgtttagtatgggcgtgc

SEQ ID NO: 46 - DNA, 30 bp, Unknown
  candidate forward primer
tccagattatattaaaatggtgtcagaacc

SEQ ID NO: 47 - DNA, 27 bp, Unknown
  candidate reverse primer
gacccagagcctttaatgtataaatcg

SEQ ID NO: 48 - DNA, 29 bp, Unknown
  candidate probe
  misc_feature (1)..(1): CY5 fluorescent dye attached
  misc_feature (29)..(29): BHQ2 non-fluorescent dye attached
acattttcaccaacagcaccagccctatt